(19) United States
(12) Patent Application Publication
(10) Pub. No.: US 2005/0124586 A1
(43) Pub. Date: Jun. 9, 2005

(54) Alpha-substituted arylalkyl phosphonate derivatives

(76) Inventors: Hieu Trung Phan, Tannay (CH); Lan Mong Nguyen, Nyon (CH); Raymond Azoulay, Geneve (CH); Vinh Van Diep, Vetraz-Monthoux (FR); Harald Eschenhof, Geneva (CH); Eric Joseph Niesor, Nyon (CH); Craig Leigh Bentzen, Bogis-Bossey (CH); Robert John Ife, Stevenage, Hertfordshire (GB)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/503,989

(22) PCT Filed: Feb. 3, 2003
(86) PCT No.: PCT/US03/03107

Related U.S. Application Data
(60) Provisional application No. 60/355,865, filed on Feb. 11, 2002.

(51) Int. Cl. A61K 31/675; A61K 31/401; A61K 31/366

(52) U.S. Cl. 514/89; 546/22; 514/423; 514/460; 514/548

(57) ABSTRACT

The present invention relates to novel α-substituted arylalkylphosphonate derivatives and their uses for lowering plasma levels of apo (a), Lp(a), apo B, apo B associated lipoproteins (low density lipoproteins and very low density lipoproteins) and for lowering plasma levels of total cholesterol.

### FIELD OF THE INVENTION

This invention relates to substituted arylalkylphosphonate compositions and therapeutic uses thereof. More specifically, the present invention relates to novel α-substituted arylalkylphosphonate derivatives, processes for their preparation, pharmaceutical compositions containing them and their use in therapy for lowering plasma levels of apo (a) and apo (a) associated lipoprotein (lipoprotein(a) or “Lp(a)”), for lowering plasma levels of apo B and apo B associated lipoproteins (low density lipoproteins and very low density lipoproteins), and for lowering plasma levels of total cholesterol.

### BACKGROUND OF THE INVENTION

Lp(a) is a LDL-like lipoprotein wherein the major lipoprotein, apo B-100, is covalently linked to an unusual glycoprotein, apoprotein(a). The covalent association between apo(a) and apo B to form Lp(a) is a secondary event which is independent of the plasma concentration of apo B. Due to its structural similarity to plasminogen, apo(a) interferes with the normal physiological thrombosis-hemostasis process by preventing thrombolysis, that is clot dissolution (see e.g., Biemond B J, Circulation 1997, 96(5) 1612-1615). The structural feature of Lp(a), where the LDL lipoprotein is linked to apo(a), is thought to be responsible for its atherogenic and thrombogenic activities.

Elevated levels of Lp(a) have been associated with the development of atherosclerosis, coronary heart disease, myocardial infarction, cerebral infarction, restenosis following balloon angioplasty and stroke. A recent epidemiologic study has provided the clinical proof of a positive correlation between plasma Lp(a) concentrations and the incidence of heart disease (A. G. Bostom, et al., Journal of American Medical Association 1996, 276, p. 544-548).

Patients that have Lp(a) levels in excess of 20-30 mg/dl run a significantly increased risk of heart attacks and stroke. An effective therapy for lowering Lp(a) does not exist at present because cholesterol lowering agents such as the HMGCoA reductase inhibitors do not lower Lp(a) plasma concentrations. The only compound that lowers Lp(a) is niacin, but the high doses necessary for activity are accompanied with unacceptable side-effects. There is, therefore, an unmet therapeutic need for agents that effectively reduce elevated levels of Lp(a).

International applications WO 97/20307, WO 98/28310, WO 98/28311 and WO 98/28312 (Symphar, SmithKline Beecham) describe a series of α-amino phosphonates which have Lp(a) lowering activity. There however remains the need to identify further compounds having Lp(a) lowering activity.

### SUMMARY OF THE INVENTION

The present invention provides, in a first aspect, a compound of formula (Ia): 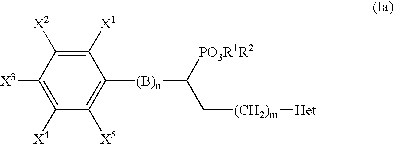
or a compound of formula (Ib): 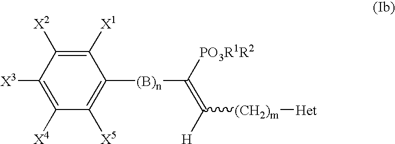
in which:
X¹, X², X³, X⁴ and X⁵ are independently hydrogen, hydroxy, hydroxymethyl, C₁-C₃ alkoxymethyl, straight or branched C₁-C₈ alkyl, straight or branched C₁-C₈ alkoxy, C₃-C₆ cycloalkyl, C₃-C₆ cycloalkoxy, cyano, halogen (F, Cl, Br, 1), and nitro; or X² may be combined with X³, or X⁴ may be combined with X⁵, to form a 5- to 6-membered alkylidenedioxy ring optionally substituted with a C₁-C₄ alkyl group; X⁴ may be combined with X⁵ to form a 5- to 6-membered alkylidene ring optionally substituted with a C₁-C₄ alkyl group;
R¹ and R² are independently hydrogen or a straight or branched C₁-C₆ alkyl;
B is CH₂, CH₂—CH₂ or CH═CH;
n is zero or 1;
m is zero, 1 or 2;
Het is an optionally substituted heteroaryl group comprising at least one nitrogen atom;
   or a pharmaceutically acceptable salt thereof.

The compound of formula (Ib) may be the Z-isomer, formula (Ib^{Z}): 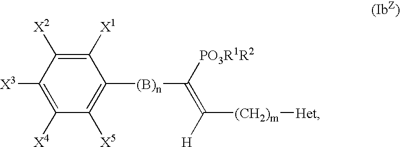
or the E-isomer, formula (Ib^{E}): 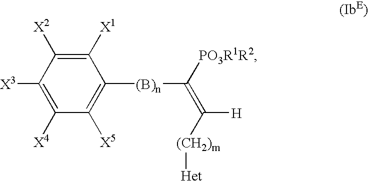
or a mixture thereof.

Compounds of the present invention include:
dimethyl α-(3,5-dimethoxy-4-hydroxyphenyl)-β-(3-pyridyl)ethylphosphonate;
diethyl α-(3,5-dimethoxy-4-hydroxyphenyl)-β-(3-pyridyl)ethylphosphonate;
diisopropyl α-(3,5-dimethoxy-4-hydroxyphenyl)-β-(3-pyridyl) ethylphosphonate;
diethyl α-(3,5-dimethoxy-4-hydroxyphenyl)-β-(5-(2-methylpyridyl)) ethylphosphonate;
diethyl α-(3,5-dimethoxy-4-hydroxyphenyl)-β-(3-(2-methylpyridyl) ethylphosphonate;
diethyl α-(3,5-dimethoxy-4-hydroxyphenyl)-β-(3-(2,6-dimethylpyridyl) ethylphosphonate;
diethyl α-(3,5-dimethyl-4-hydroxyphenyl)-β-(3-pyridyl)ethylphosphonate;
dimethyl α-(4-hydroxy-3-methoxy-5-methylphenyl)-β-(3-pyridyl)ethylphosphonate;
diethyl α-(4-hydroxy-3-methoxy-5-methylphenyl)-β-(3-pyridyl) ethylphosphonate;
diisopropyl α-(4-hydroxy-3-methoxy-5-methylphenyl)-β-(3-pyridyl) ethylphosphonate;
dimethyl α-(4-hydroxy-3-methoxy-5-methylphenyl)-β-(5-(2-methylpyridyl)) pyridyl)ethylphosphonate;
diethyl α-(4-hydroxy-3-methoxy-5-methylphenyl)-β-(5-(2-methylpyridyl)) ethylphosphonate;
diisopropyl α-(4-hydroxy-3-methoxy-5-methylphenyl)-β-(5-(2-methylpyridyl)) ethylphosphonate;
dimethyl α-(4-hydroxy-3-methoxy-5-methylphenyl)-β-(3-(2-methylpyridyl)) pyridyl)ethylphosphonate;
diethyl α-(4-hydroxy-3-methoxy-5-methylphenyl)-β-(3-(2-methylpyridyl)) ethylphosphonate;
diethyl α-(4-hydroxy-3-methoxy-5-methylphenyl)-β-(3-(2,6-dimethylpyridyl)) ethylphosphonate;
diethyl α-(4-hydroxy-3-methoxy-5-methylphenyl)-β-(4-(3,5-dimethylisoxazolyl))ethylphosphonate;
diethyl α-(4-hydroxy-3-methoxy-5-methylphenyl)-β-(4-(2-methylthiazolyl)) ethylphosphonate;
diethyl α-(4-hydroxy-3-methoxy-5-methylphenyl)-β-(pyrazinyl) ethylphosphonate;
E)-diisopropyl α-(3,5-dimethoxy-4-hydroxyphenyl)-β-(3-pyridyl) vinylphosphonate;
(E)-diisopropyl α-(4-hydroxy-3-methoxy-5-methylphenyl)-β-(3-pyridyl) vinylphosphonate;
(E)-diisopropyl α-(4-hydroxy-3-methoxy-5-methylphenyl)-β-(5-(2-methylpyridyl)) vinylphosphonate;
(E)-diethyl α-(3,5-di-tert-butyl-4-hydroxyphenyl)-β-(3-pyridyl)) ethylphosphonate;
(Z)-(diethyl α-(3,5-tert-butyl-4-hydroxybenzyl)-β-(3-pyridyl) vinylphosphonate;
(E)-diisopropyl α-(3,5-dimethoxy-4-hydroxybenzyl)-β-(3-pyridyl)vinyl phosphonate; and
diisopropyl α-(3,5-dimethoxy-4-hydroxybenzyl)-β-(3-pyridyl)ethylphosphonate.

One aspect of the present invention provides for a pharmaceutical composition comprising a compound of formula (Ia) or formula (Ib) and a pharmaceutically acceptable excipient. Hereinafter compounds of formula (Ia) and compounds of formula (Ib) are collectively termed “compounds of formula (I).”

The present invention also provides for therapeutic uses of the compounds of formula (I). In one aspect, the invention provides for a method of decreasing plasma levels of apo (a) and lipoprotein(a), in reducing plasma levels of apo B and LDL cholesterol and in decreasing plasma total cholesterol. The present invention also provides further methods including: a method of prevention and/or treatment of thrombosis by increasing thrombolysis through decreasing plasma levels of apo (a) and lipoprotein(a); a method of treatment of restenosis following angioplasty by decreasing plasma levels of apo (a) and lipoprotein(a); a method of prevention and/or treatment of atherosclerosis by decreasing plasma levels of apo (a) and lipoprotein(a) or by decreasing plasma levels of apoprotein B and LDL cholesterol; a method of prevention and/or treatment of hypercholesterolemia; a method of prevention and/or treatment of atherosclerosis by lowering cholesterol in patients that are resistant to treatment with statins; and a method of prevention and/or treatment of atherosclerosis in association with a compound such as a statin which decreases cholesterol synthesis.

### DETAILED DESCRIPTION OF THE INVENTION

The present invention relates to the compounds of formula (I) and their uses for lowering plasma levels of apo (a), Lp(a), apo B, apo B associated lipoproteins (low density lipoproteins and very low density lipoproteins) and for lowering plasma levels of total cholesterol.

In relation to compounds of formula (1), in preferred embodiments, X¹ is hydrogen, or methyl, X² is methoxy, ethoxy, methyl, tert-butyl or hydroxy, X³ is hydrogen, hydroxy, methoxy, methyl, ethyl or hydroxymethyl, X⁴ is hydrogen, methoxy, methyl or tert-butyl and X⁵ is hydrogen. In a preferred combination, X² is methoxy, X³ is hydroxy and X⁴ is methyl or methoxy. Preferably, n is zero, so that (B), is replaced with a direct bond. Preferably R¹ and R² are C₁-C₃ alkyl, more preferably C₂ or C₃, and in particular wherein R¹ and R² are independently ethyl or isopropyl. Preferably m is zero.

When used herein the term “heteroaryl” refers to, unless otherwise defined, a single or a fused ring containing up to four heteroatoms in each ring, each of which is selected from oxygen, nitrogen and sulphur, which rings may be unsubstituted or substituted by, for example, up to four substituents. Each ring suitably has from 4 to 7, preferably 5 or 6 ring atoms. A fused ring system may include carbocyclic rings and need include only one heteroaryl ring.

Representative examples of Het include pyridyl, pyrimidyl, pyrazinyl, pyridazinyl, thiazolyl, thiadiazolyl, benzothiazolyl, isoxazolyl, pyrazolyl, triazinyl, and imidazolyl which may be unsubstituted or substituted by up to four substituents (for pyridyl and benzothiazolyl), three substituents (pyrimidyl, pyrazinyl, pyridazinyl, pyrazolyl), two substituents (thiazolyl, isoxazolyl, triazinyl and imidazolyl) or one substituent (thiadiazolyl) which may be the same or different and selected from straight or branched C₁-C₄ alkyl or alkoxy, hydroxy, hydroxymethyl, halogen (F, Cl, Br, I), or an amino group optionally substituted with C₁-C₄ alkyl. Preferably, pyridyl, pyrimidyl, pyrazinyl, pyridazinyl, thiazolyl, thiadiazolyl, benzothiazolyl, pyrazolyl, or triazinyl is unsubstituted or substituted by methyl, methoxy, dimethoxy or dimethyl. Preferred examples of Het include 3-pyridyl, 3-(2-methylpyridyl), 3-(5-methylpyridyl), 3-(2,6-dimethylpyridyl), 2-pyranizyl, 4-(3,5-diemthylisoxazoyl) or 4-2-methylthiazolyl).

Pharmaceutically acceptable salts for use in the present invention include those described by Berge, Bighley, and Monkhouse, J. Pharm. Sci., 1977, 66, 1-19. Such salts may be formed from inorganic and organic acids. Representative examples thereof include maleic, fumaric, benzoic, ascorbic, pamoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, benzenesulfonic, hydrochloric, hydrobromic, sulfuric, cyclohexylsulfamic, phosphoric and nitric acids.

It will be appreciated that certain compounds of the present invention, in particular those of formula (Ia), will comprise one or more chiral centres so that compounds may exist as stereoisomers, including diastereoisomers and enantiomers. The present invention covers all such stereoisomers, and mixtures thereof, including racemates. The compounds of formula (Ib) of the present invention comprise the individual E- and Z-diastereoisomers and mixtures thereof.

Since the compounds of the present invention are intended for use in pharmaceutical compositions, it will be understood that they are each provided in substantially pure form, for example at least 50% pure, more suitably at least 75% pure and preferably at least 95% pure (% are on a wt/wt basis). Impure preparations of the compounds of formula (I) may be used for preparing the more pure forms used in the pharmaceutical compositions. Although the purity of intermediate compounds of the present invention is less critical, it will be readily understood that the substantially pure form is preferred as for the compounds of formula (I). Preferably, whenever possible, the compounds of the present invention are obtained in crystalline form.

When some of the compounds of this invention are allowed to crystallise or are recrystallised from organic solvents, solvent of crystallisation may be present in the crystalline product. This invention includes within its scope such solvates. Similarly, some of the compounds of this invention may be crystallised or recrystallised from solvents containing water. In such cases water of hydration may be formed. This invention includes within its scope stoichiometric hydrates as well as compounds containing variable amounts of water that may be produced by processes such as lyophilisation. In addition, different crystallisation conditions may lead to the formation of different polymorphic forms of crystalline products. This invention includes within its scope all polymorphic forms of the compounds of formula (I).

The present invention also relates to the unexpected discovery that compounds of formula (I) are effective for decreasing apo(a) production in vitro and Lp(a) production in vivo in Cynomolgus monkeys. This species has been selected as the animal model as its Lp(a) is similar in immunologic properties to human Lp(a) and occurs in almost identical frequency distribution of plasma concentrations, see e.g., N. Azrolan et al; J. Biol. Chem., 266, 13866-13872 (1991). In the in vitro assay, compounds of formula (I) have been shown to reduce the secretion of apo (a) which is secreted in free form from the primary cultures of the Cynomolgus monkey hepatocytes. These results are confirmed by the in vivo studies performed on the same animal species showing the potent decrease of Lp(a) by compounds of formula (I). Therefore the compounds of this invention are useful for decreasing apo (a) and Lp(a) in man and thus provide a therapeutic benefit.

Accordingly in a further aspect, this invention provides a compound of formula (I) or a pharmaceutically acceptable salt thereof for use in therapy, in particular as a Lp(a) lowering agent. Elevated plasma and tissue levels of Lp(a) are associated with accelerated atherosclerosis, abnormal proliferation of smooth muscle cells and increased thrombogenesis and expressed in disease states such as, for instance: coronary heart disease, peripheral artery disease, intermittent claudication, thrombosis, restenosis after angioplasty, extra-cranial carotid atherosclerosis, stroke and atherosclerosis occurring after heart transplantation.

Furthermore, the compounds of the present invention may possess cholesterol lowering properties and decrease total plasma cholesterol, in particular LDL cholesterol. It is now well established that a high level of LDL cholesterol is a major risk factor for atherosclerotic diseases. In addition, the compounds of the present invention may decrease the levels of apoprotein B (apo B) which is the main protein of LDL and the main ligand for LDL receptors. The mechanism of decrease in apo B and in apo B-associated LDL probably does not involve inhibition of cholesterol synthesis, which is the mechanism demonstrated for the statins. Therefore, compounds of the present invention are useful for lowering cholesterol in patients who are resistant to treatment with a statin, and, conversely, also have an additive or synergistic effect for lowering cholesterol in those patients who are responding to treatment with statins. Thus, compounds of the present invention are of use in therapy as cholesterol lowering agents. Furthermore, a dual profile in lowering plasma Lp(a) and plasma cholesterol makes the compounds of formula (I) useful in therapy for the prevention and/or treatment of both the acute and chronic aspects of atherosclerosis.

Compounds of the present invention may also be of use in preventing and/or treating the above mentioned disease states in combination with anti-hyperlipidaemic, anti-atherosclerotic, anti-diabetic, anti-anginal, anti-inflammatory or anti-hypertension agents. Examples of the above include cholesterol synthesis inhibitors such as statins, for instance atorvastatin, simvastatin, pravastatin, cerivastatin, fluvastatin, lovastatin and ZD 4522 (also referred to as S-4522, Astra Zeneca), anti-oxidants such as probucol, insulin sensitisers such as a PPAR gamma activator, for instance G1262570 (Glaxo Wellcome) and the glitazone class of compounds such as rosiglitazone (Avandia, SmithKline Beecham), troglitazone and pioglitazone, calcium channel antagonists, and anti-inflanmmatory drugs such as NSAIDs.

For therapeutic use the compounds of the present invention will generally be administered in a standard pharmaceutical composition. Accordingly in a further aspect, the invention provides for a pharmaceutical composition comprising a compound of formula (I) or a pharmaceutically acceptable salt thereof and a pharmaceutically acceptable excipient or carrier. Suitable excipients and carriers are well known in the art and will be selected with regard to the intended route of administration and standard pharmaceutical practice. For example, the compositions may be administered orally in the form of tablets containing such excipients as starch or lactose, or in capsules, ovules or lozenges either alone or in admixture with excipients, or in the form of elixirs or suspensions containing flavoring or coloring agents. They may be injected parenterally, for example, intravenously, intramuscularly or subcutaneously. For parenteral administration, they are best used in the form of a sterile aqueous solution which may contain other substances, for example, enough salts or glucose to make the solution isotonic with blood. The choice of form for administration as well as effective dosages will vary depending, inter alia, on the condition being treated. The choice of mode of administration and dosage is within the skill of the art.

The compounds of formula (I) and their pharmaceutically acceptable salts which are active when given orally can be formulated as liquids, for example syrups, suspensions or emulsions or as solids for example, tablets, capsules and lozenges. A liquid formulation will generally consist of a suspension or solution of the compound or pharmaceutically acceptable salt in suitable liquid carrier(s) for example, ethanol, glycerine, non-aqueous solvent, for example polyethylene glycol, oils, or water with a suspending agent, preservative, flavoring or coloring agents. A composition in the form of a tablet can be prepared using any suitable pharmaceutical carrier(s) routinely used for preparing solid formulations. Examples of such carriers include magnesium stearate, starch, lactose, sucrose and cellulose. A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration. A typical suppository formulation comprises a compound of structure (I) or a pharmaceutically acceptable salt thereof which is active when administered in this way, with a binding and/or lubricating agent such as polymeric glycols, gelatins or cocoa butter or other low melting vegetable or synthetic waxes or fats. Preferably the composition is in unit dose form such as a tablet or capsule.

Each dosage unit for oral administration contains preferably from 1 to 250 mg (and for parenteral administration contains preferably from 0.1 to 25 mg) of a compound of formula (I) or a pharmaceutically acceptable salt thereof calculated as the free base.

The compounds of the invention will normally be administered to a subject in a daily dosage regimen. For an adult patient this may be, for example, an oral dose of between 1 mg and 500 mg, preferably between 1 mg and 250 mg, or an intravenous, subcutaneous, or intramuscular dose of between 0.1 mg and 100 mg, preferably between 0.1 mg and 25 mg, of the compound of the formula (I) or a pharmaceutically acceptable salt thereof calculated as the free base, the compound being administered 1 to 4 times per day.

The present invention also relates to processes for preparing novel α-substituted arylalkylphosphonate derivatives of formula (1), which is described below.

Compounds of formula (Ib) may be prepared by a process which comprises condensing a phenylalkylphosphonate of formula (II): 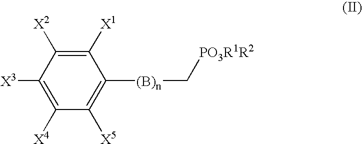
in which X¹, X², X³, X⁴, X⁵, B, n, R¹ and R² are as previously defined; with an aldehyde of formula (III): 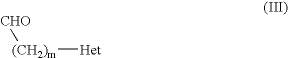
in which m and Het are as previously defined.

The condensation reaction between (II) and (III) can be carried out in several ways. In the first variant the α silyl carbanion of the phenylalkylphosphonate (II) is condensed with the aldehyde (III) under the conditions of the Peterson olefination reaction. Suitable silylating reagents include chlorotrimethylsilane or chlorotriethylsilane. A preferred silylating agent is chlorotrimethylsilane. Suitably, the condensation may be carried out in an ether solvent such as diethyl ether, tetrahydrofuran (THF), dimethoxyethane or dioxane. A preferred solvent is THF. Suitable bases include n-butyllithium, lithium diisopropylamide (LDA) formed in situ by reacting n-butyllithium and diisopropylamine, or n-buthyllithium used in association with N,N,N′,N′-tetramethylethylenediamine. The reaction is suitably carried out in the range from −78° C. to room temperature (20° C.).

Another variant consists in reacting the carbanion of the phenylalkyldiphosphonate (IV) 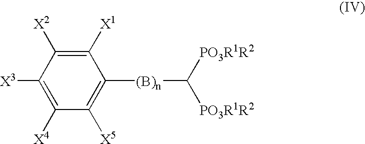
with the aldehyde (III) under the Horner-Emmons olefination reaction. Suitably, the condensation may be carried out in an ether solvent such as diethyl ether, tetrahydrofuran (THF), dimethoxyethane, dioxane, or dimethylformamide (DMF). A preferred solvent is THF. Suitable bases include sodium hydride, n-butyllithium, lithium diisopropylamide (LDA) formed in situ by reacting n-butyllithium and diisopropylamine, or n-butyllithium used in association with TMEDA (N,N,N′,N′-tetramethylethylenediamine) The reaction is suitably carried out in the range from −78° C. to room temperature (20° C.).

Both of these two mentioned variants of the condensation of a phenylalkylphosphonate of formula (II) or a phenylalkyldiphosphonate of formula (IV) with an aldehyde of formula (III) afford compounds of formula (Ib). The two isomers (Ib^{Z}) and (Ib^{E}) can be separated by column chromatography. The structures of these isomers are ascertained by spectroscopic means: MS and in particular NMR, thanks to the characteristic absorption of the olefinic proton. In the (Z)-isomer (Ib^{z}), the olefinic proton displays a large coupling constant, J=ca 40-43 Hz, due to the trans H—C═C—P coupling. In the (E)-isomer (Ib^{E}) this value is much smaller, J=ca 25 Hz, due to the cis H—C═C—P coupling.

Compounds of formula (Ia) can be prepared by reducing compounds of formula (Ib) either as a mixture of both isomers or from the isomers of formula (Ib^{Z}) or formula (IB^{E}):

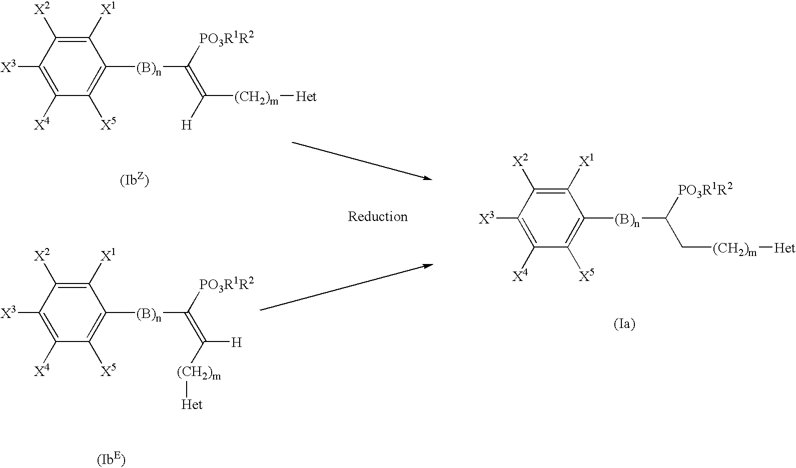

A suitable reduction method is the catalytic hydrogenation using as catalysts palladium or platinum adsorbed on charcoal in a solvent such as ethanol or acetic acid at a pressure between 1 and 4 atm and a temperature between room temperature and 40° C. The reduction can also be carried out by means of a complex hydride reagent such as sodium borohydride or sodium cyanoborohydride in a polar solvent such as methanol, ethanol, isopropanol or n-propanol at a temperature between room and reflux temperature. A further convenient reduction method is the use of a zinc modified sodium cyanoborohydride reagent generated from a mixture of NaBH₃CN:ZnCl₂ in a 2:1 molar ratio in a solvent selected from diethyl ether, tetrahydrofulran, dimethoxyethane and methanol at a temperature between room temperature and reflux temperature; the reaction can be accelerated by the addition of a higher boiling solvent selected from ethanol, isopropanol, n-propanol, isobutanol or n-butanol and heating to reflux the resulting mixture.

In a further variant, compound (Ia) can be directly obtained by the reaction between the phenyalkylphosphonate (II) and an alkyl halide of formula (V), 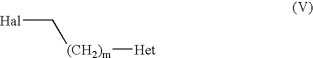
wherein Hal is Cl or Br, in presence of a base. Suitable solvents include diethyl ether, tetrahydrofuran (THF), dimethoxyethane or dioxane. A preferred solvent is THF. Suitable bases include n-butyllithium, lithium diisopropylamide (LDA) formed in situ by reacting n-butyllithium and diisopropylamine, or n-butyllithium used in association with TMEDA The reaction is suitably carried out in the range from −78° C. to room temperature (20° C.).

When any of the substituents X¹, X², X³, X⁴, X⁵ is a hydroxy group, giving a reactive phenol or hydroxymethylphenyl group, it may be useful to protect such a hydroxy group, to avoid troublesome side reactions which may otherwise occur under the strongly alkaline reaction conditions employed. A particularly effective way of protecting the OH group is to convert it into an alkyl silyl ether, such as trimethyl silyl ether (Me₃Si ether or Tms ether) or a t-butyldimethyl silyl ether (tBuMe₂Si ether or Tbs ether). An integral part of this invention is the conversion of a phosphonate of formula (II) or (IV) comprising a hydroxy group into the corresponding Tbs ether. Suitable protection reaction conditions are the use of t-butyldimethylsilyl chloride in presence of imidazole in dimethylformamide. Such an Tbs protected phosphonate (D) or diphosphonate (W) can then withstand the strongly alkaline conditions which are necessary to form the desired Tbs-protected (Ia) or (Ib) structures. The Tbs protecting group can then be cleaved by fluoride reagents well established in the art to yield the end products of formula (I) wherein any of the substituents X¹, X², X³, X⁴, X⁵ can be a hydroxy group. Suitable deprotection reaction conditions involve reacting the Tbs protected compound with tetrabutyl ammonium fluoride in THF in the presence of glacial acetic acid.

The various starting compounds phenylalkylphosphonates (II), phenylalkyldiphosphonates (IV), aldehydes (III) and halide (V) can be prepared according to methods described in the chemical literature.

### EXAMPLES OF THE INVENTION

The invention is further described in the following examples that are intended to illustrate the invention without limiting its scope. The abbreviations used in this application are the following: in the tables, n is normal, i is iso, s is secondary and t is tertiary. In the description of the NMR spectra, respectively s is singlet, d doublet, dd double doublet, t triplet, q quadruplet and m multiplet. The temperatures were recorded in degrees Celsius and the melting points are not corrected.

The structures of compounds described in the Examples were established by their infrared (IR), mass (MS) and nuclear magnetic resonance (NMR) spectra. The purity of the compounds was checked by thin layer, gas, liquid or high performance liquid chromatography.

Unless otherwise indicated, the physical constants and biological data given for compounds of formula (Ia) refer to racemates while those given for compounds of formula (Ib^{E}) and (Ib^{Z}) refer to pure isomers.

### Example 1

### Diethyl α-(3,5-dimethoxy-4-hydroxyphenyl)-β-(3-pyridyl) Ethylphosphonate

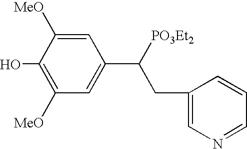

Imidazole (10 g, 14.8 mmol) was added portionwise to a well stirred mixture of 4-hydroxy-3,5-dimethoxybenzylphosphonate (14 g, 46 mmol) and t-butyldimethylsilyl chloride (9 g, 60 mmol) in 80 ml N,N-dimethylformamide (DMF) and stirring was continued for 16 h at room temperature. The mixture was poured into water kept at 0° C. to which was added a 25% ammonium hydroxide solution until pH 7 was reached. The aqueous phase was extracted with dichloromethane, the organic phase was dried over MgSO₄. Evaporation of the solvent gave 17 g (89%) of diethyl (4-t-butyldimethylsilyloxy-3,5-dimethoxybenzyl)phosphonate as a dark oil.

A solution of diethyl (4-t-butyldimethylsilyloxy-3,5-dimethoxybenzyl) phosphonate (7 g, 16.7 mmol) in 40 ml THF was added dropwise to a solution of nBuLi 1.6 M (41 ml, 66.8 mmol) in 80 ml THF kept at −78° C. After 30 min a suspension of 3-(chloromethyl)pyridine hydrochloride (5.5 g, 33.4 mmol) in 30 ml THF was added dropwise (int. temp. ≦−70°) and stirring was continued at −78° for 1 h. The cooling bath was removed and the reaction mixture was allowed to warm to room temperature. After 2 h at room temperature the mixture was cooled with an ice bath and H₂O (30 ml) was added dropwise. Concentration in vacuo gave an emulsion which was partitioned between 40 ml saturated NaCl solution and 250 ml CHCl₃. The aqueous layer was separated and extracted with two further portions of 250 ml CHCl₃. The combined organic phases were dried with MgSO₄ and evaporated to afford 8.4 g of a brown oil. Purification of this residue by column chromatography (CH₂Cl₂/MeOH 95/5) yielded 3.4 g (6.7 mmol, 40%) of diethyl α-(4-t-butyldimethylsilyloxy-3,5-dimethoxy)-β-(3-pyridyl)ethylphosphonate as a yellow oil.

A solution of tetrabutylammonium fluoride (8.3 g, 26.6 mmol) in 190 ml THF was added in one portion to a solution of the preceding compound (3.4 g, 6.7 mmol) in 90 ml THF. The reaction solution was stirred at room temperature for 3 h and was partitioned between 11 CH₂Cl₂ and 100 ml H₂O. The organic phase was separated and washed with 21 saturated NaHCO₃ solution. Drying with MgSO₄ and evaporation gave 2.1 g of a brown oil. This crude product was purified by column chromatography (CH₂Cl₂/MeOH 9:1) furnishing 0.65 g (1.6 mmol, 25%) of a yellow oil which gave colourless crystals, m.p. 104-107°, after trituration in hexane.

MS (m/e)=395: M⁺, 258: M⁺-PO₃Et₂

NMR (CDCl₃):

δ=8.38, 8.33, 7.25 and 7.08 (4m, 1H each): aromatic H, 3-pyridyl 6.48 (d, J=2 Hz, 2H): aromatic H, substituted phenyl 5.71 (s, 1H): OH4.15-3.65 (m, 4H): P—O—CH₂-CH₃ 3.81 (s, 6H): Ph-OCH₃ 3.42-3.39 (m, 1H): (Ph)(P)CH—CH₂-pyridine 3.19-3.08 (m, 2H): (Ph)(P)CH—CH₂-pyridine 1.31 and 1.11 (2t, 1H each, J=7 Hz): P—O—CH₂—CH₃

### Example 2

### Diethyl α-(3,5-dimethoxy-4-hydroxyphenyl)-β-(5-(2-methylpyridyl)) ethylphosphonate

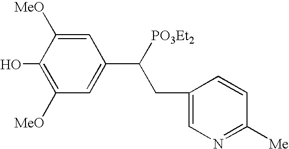

A solution of methyl 6-methylnicotinate (25.0 g, 165 mmol) in 50 ml dry ether was added dropwise to a vigorously stirred suspension of LiAlH₄ (9.41 g, 248 mmol) in 325 ml dry ether. The reaction mixture was heated to reflux with the oil bath of 55° for 1.5 h and was then cooled to 0°. Water (45 ml) was added dropwise and, 1 h later, the upper layer was decanted off. The remaining suspension was extracted with ether (9 portions of 250 ml). The combined organic phases were dried with MgSO₄ and evaporated to yield 19.7 g (160 mmol, 97%) of 5-(hydroxymethyl)-2-methylpyridine as an orange oil; GC-analysis indicated a purity of 98%.

A solution of this alcohol compound (52.2 g, 424 mmol) in 190 ml toluene and 60 ml CHCl₃ was added dropwise to a solution of SOCl₂ (34 ml, 469 mmol) in 44 ml toluene, all the while maintaining the internal temperature between 23° and 35°. After the end of the addition the reaction mixture was vigorously stirred at room temperature for 1 h and water pump vacuum was applied until the solvent was completely evaporated. The brown precipitate was resuspended in toluene, rapidly filtered off and washed three times with toluene. Drying in the desiccator (aspirator vacuum) gave 72.1 g (405 mmol, 96%) of 5-(chloromethyl)-2-methylpyridine hydrochloride as a brown solid. This hydrochloride (3.86 g, 21.7 mmol) was partitioned between 70 ml CH₂Cl₂ and 8 ml NaOH 10%. The aqueous phase (pH 7-8) was separated and extracted with another portion of 70 ml CH₂Cl₂. The combined organic phases were dried with MgSO₄ and evaporated to yield 2.73 g (19.3 mmol, 89%) of 5-(chloromethyl)-2-methylpyridine as a brown oil. GC-analysis of the free base indicated a purity of 99%.

Diethyl (4-t-butyldimethylsilyloxy-3,5-dimethoxybenzyl)phosphonate (170 g, 0.41 mol) was prepared by reacting diethyl (3,5-dimethoxy-4-hydroxybenzyl) phosphonate (130 g, 0.43 mol) with t-butyldimethylsilyl chloride (96.5 g, 0.64 mol) in 400 ml N,N-dimethylformamide (DMF) in presence of imidazole (58.2 g, 0.86 mol).

A solution of diethyl (4-t-butyldimethylsilyloxy-3,5-dimethoxybenzyl)-phosphonate (4.03 g, 9.63 mmol) in 18 ml THF was added dropwise to a solution of nBuLi 1.6 M (14 ml, 22.4 mmol) in 37 ml THE kept at −78° C. After 30 min a solution of the free base of the 5-chloromethyl-2-methylpyridine (2.73 g, 19.3 mmol) in 3 ml THE was added dropwise with a syringe (int. temp. ≦−70°) and stirring was continued at −78° for 1 h. The cooling bath was removed and the reaction mixture was allowed to warm to room temperature. After 2 h at room temperature the mixture was cooled with an ice bath and H₂O (40 ml) was added dropwise. Concentration in vacuo (400 mbar→100 mbar) gave an emulsion which was partitioned between 40 ml saturated NaCl solution and 250 ml CHCl₃. The aqueous layer was separated and extracted with two further portions of 250 ml CHCl₃. The combined organic phases were dried with MgSO₄ and evaporated to afford 6.37 g of a brown oil. Purification of this residue by column chromatography (CH₂Cl₂/MeOH 19:1) yielded 2.07 g (3.95 mmol, 41%) of diethyl α-(4-t-butyldimethylsilyloxy-3,5-dimethoxy)-β-(5-(2-methylpyridyl))ethylphosphonate as a brown yellow oil; GC-analysis: 98%.

A solution of tetrabutylammonium fluoride (2.11 g, 6.69 mmol) in 190 ml THF was added in one portion to a solution of the preceding compound (14.0 g, 26.7 mmol) in 190 ml THF. The reaction solution was stirred at room temperature for 3 h and was partitioned between 1.71 CH₂Cl₂ and 130 ml H₂O. The organic phase was separated and washed with 21 saturated NaHCO₃ solution. Drying with MgSO₄ and evaporation gave 13.4 g of a brown oil. This crude product was purified by column chromatography (CH₂Cl₂/MeOH 9:1) furnishing 9.18 g (22.4 mmol, 84%) of a yellow oil. A sample of 5.17 g was crystallized from hexane/AcOEt affording 3.59 g of the title compound as colourless crystals, m.p. 100-102′; GC-analysis of crystallized product: 100%.

MS (m/e): 409: M⁺, 303: M⁺-CH₂—C₆H₆N

¹H-NMR (CDCl₃):

δ=8.20 (s, 1H): aromatic H, substituted pyridyl 7.14 (dd, J=7.9 Hz and J=2.2 Hz, 1H): aromatic H, substituted pyridyl 6.93 (d, J=7.9 Hz, 1H): aromatic H, substituted pyridyl 6.48 (s, 2H): aromatic H, substituted phenyl 5.63 (s, 1H): OH 4.08, 3.94 and 3.72 (3m, 4H total): P—O—CH₂—CH₃ 3.82 (s, 6H): Ph-OCH₃ 3.39-3.33 (m, 1H): (Ph)(P)CH—CH₂ 3.15-3.05 (m, 2H): (Ph)(P)CH—CH₂ 2.46 (s, 3H): Py-CH₃ 1.32 and 1.12 (2t, J=7.1 Hz, 6H total): P—O—CH₂—CH₃

### Example 3

### Diethyl α-(3,5-dimethoxy-4-hydroxyphenyl)-β-(3-(2,6-dimethylpyridyl)) ethyl-phosphonate

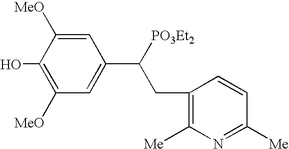

A solution of ethyl 3-aminocrotonate (46.5 g, 380 mmol) in 35 ml benzene was added very slowly to a solution of 3-butyn-2-one (25.9 g, 380 mmol) in 35 ml benzene (strong heat development) and the reaction mixture was stirred overnight. The precipitate was filtered off and washed with little benzene. Drying in the desiccator gave beige crystals (61.3 g, m.p. 122-131°). This solid was heated to 130° for 2 h while the formed water was distilled off. The remaining brown oil was diluted with CH₂Cl₂, dried with MgSO₄ and concentrated in vacuo (40-80°) to give 52.0 g of ethyl 2,6-dimethylnicotinate (290 mmol, 76%) as a brown oil (preparation according to H. Pasedach and M. Seefelder, DE 1,207,930, Dec. 30, 1965).

A solution of the previous compound (64.7 g, 361 mmol) in 500 ml dry ether was added dropwise to a vigorously stirred suspension of LiAlH₄ (20.6 g, 543 mmol) in 930 ml dry ether. The reaction mixture was heated to reflux with the oil bath of 55° for 1.5 h and was then cooled to 0°. Water (100 ml) was added dropwise and, 1 h later, the upper layer was decanted off and the remaining suspension was extracted with ether. The combined organic phases were dried with MgSO₄ and evaporated to yield 49.6 g (361 mmol, 1000%) of 5-(hydroxymethyl)-2,6-dimethyl-pyridine as an yellow oil; GC-analysis indicated a purity of 100%.

A solution of this alcohol compound (32.2 g, 235 mmol) in 144 ml toluene and 120 ml CHCl₃ was added dropwise to a solution of SOCl₂ (18.8 ml, 259 mmol) in 24 ml toluene, all the while maintaining the internal temperature between 23° and 35°. After the end of the addition the reaction mixture was vigorously stirred at 35° for 1.5 h and water pump vacuum was applied until the solvent was completely evaporated. The brown precipitate was resuspended in toluene, rapidly filtered off and washed three times with toluene. Drying in the desiccator (aspirator vacuum) gave 33.8 g (176 mmol, 75%) of 5-(chloromethyl)-2,6-dimethylpyridine hydrochloride as a light brown solid.

A solution of diethyl (4-t-butyldimethylsilyloxy-3,5-dimethoxybenzyl phosphonate (2.50 g, 5.97 mmol) in 11 ml THF was added dropwise to a solution of nBuLi 1.6 M (15 ml, 24.0 mmol) in 16 ml THF kept at −78° C. After 30 min. 5-(chloromethyl)-2,6-dimethylpyridine hydrochloride (2.29 g, 11.9 mmol) was added portionwise over 15 min. (int. temp. ≦−70°) and stirring was continued at −78° for 1 h. The cooling bath was removed and the reaction mixture was allowed to warm to room temperature. After 2 h at room temperature the mixture was cooled with an ice bath and H₂O (18 ml) was added dropwise. Concentration in vacuo gave an emulsion which was partitioned between saturated NaCl solution and CHCl₃. The aqueous layer was separated and extracted with two further portions of 150 ml CHCl₃. The combined organic phases were dried with MgSO₄ and evaporated to afford 4.28 g of a brown oil. Purification of this residue by column chromatography (CH₂Cl₂/MeOH 19:1) yielded 1.13 g (2.16 mmol, 36%) of diethyl α-(4-t-butyldimethylsilyloxy-3,5-dimethoxy)-β-(5-(2,6-dimethylpyridyl))ethylphosphonate as a yellow oil; GC-analysis: 92%.

Acetic acid (1.48 ml, 25.9 mmol) was added to a solution of the preceding compound (1.13 g, 2.16 mmol) and tetrabutylammonium fluoride (2.73 g, 8.65 mmol) in 29 ml THF. The reaction solution was stirred at room temperature for 3 h and was cooled with the ice bath. 23 ml NaOH 10% were added dropwise and the mixture was extracted with CH₂Cl₂. The organic phase was washed with saturated NaHCO₃ solution, dried with MgSO₄ and evaporated to give 1.12 g of a red oil. This residue was purified by column chromatography (CH₂Cl₂/MeOH 9:1) furnishing 770 mg (1.82 mmol, 84%) of a slightly yellowish oil; GC-analysis: 96%.

MS (m/e): 423: M⁺, 303: M⁺-CH₂—C₇H₈N, 121 (100%)

¹H-NMR (CDCl₃):

δ=6.93, 6.73 (2d, 1H each, J=7.8 Hz each): aromatic H, substituted pyridyl 6.47 (d, 2H, 1.5 Hz): aromatic H, substituted phenyl 5.64 (s, 1H): OH 4.01, 3.92 and 3.68 (3m, 4H total): P—O—CH₂—CH₃ 3.81 (s, 6H): Ph-OCH₃ 3.44-3.38 (m, 1H): (Ph)(P)CH—CH₂ 3.08-2.99 (m, 2H): (Ph)(P)CH—CH₂ 2.49 and 2.43 (2s, 6H total): Py-CH₃ 1.33 and 1.10 (2t, J=7.0 Hz, 6H total): P—O—CH₂—CH₃

### Example 4

### Diethyl α-(4-hydroxy-3-methoxy-5-methylphenyl)-β-(3-pyridyl)ethyl phosphonate

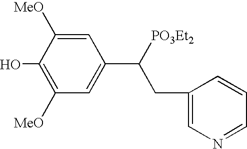

A solution of diethyl (4-t-butyldimethylsilyloxy-3-methoxy-5-methylbenzyl) phosphonate (5.00 g, 12.4 mmol) in 21 ml THF was added dropwise to a solution of nBuLi 1.6 M (31 ml, 49.6 mmol) in 31 ml THF kept at −78° C. After 30 min. 3-(chloromethyl) pyridine hydrochloride (4.07 g, 24.8 mmol) was added portionwise over 15 min. (int. temp. ≦−70°) and stirring was continued at −78° for 1 h. The cooling bath was removed and the reaction mixture was allowed to warm to room temperature. After 24 h at room temperature the mixture was cooled with an ice bath and H₂O (37 ml) was added dropwise. Concentration in vacuo (400 mbar→100 mbar) gave an emulsion which was partitioned between saturated NaCl solution and CHCl₃. The aqueous layer was separated and extracted with CHCl₃. The combined organic phases were dried with MgSO₄ and evaporated to afford 8.70 g of a red-brown oil. Purification of this residue by column chromatography (CH₂Cl₂/MeOH 19:1) yielded 2.48 g (5.03 mmol, 41%) of diethyl α-(4-t-butyldimethylsilyloxy-3-methoxy-5-methylphenyl)-β-(3-pyridyl) ethylphosphonate as a yellow oil; GC-analysis: 97%.

Acetic acid (3.45 ml, 60.3 mmol) was added to a solution of the previous compound (2.48 g, 5.03 mmol) and tetrabutylammonium fluoride (6.34 g, 20.1 mmol) in 68 ml THF. The reaction solution was stirred at room temperature for 3 h and was cooled with the ice bath. 23 ml NaOH 10% were added dropwise and the mixture was extracted with CH₂Cl₂. The organic phase was washed with saturated NaHCO₃ solution, dried with MgSO₄ and evaporated to give 2.44 g of a yellow-brown oil. This residue was purified by column chromatography (CH₂Cl₂/MeOH 9:1) furnishing 1.67 g (4.40 mmol, 88%) of a yellowish oil; GC-analysis: 100%.

MS (m/e): 379: M⁺, 287: M⁺-CH₂—C₅H₄N

¹H-NMR (CDCl₃):

δ=8.37 and 8.31 (2d, 4.6 Hz and 1.1 Hz, 1H each): aromatic H, pyridyl 7.29 (d, 7.9 Hz, 1H): aromatic H, pyridyl 7.08 (m, 1H): aromatic H, pyridyl 6.65 and 6.58 (2s, 2H total): aromatic H, substituted phenyl 5.77 (s, 1H): OH 4.08, 3.93, and 3.71 (3m, 4H total): P—O—CH₂—CH₃ 3.81 (s, 3H): Ph-OCH₃ 3.34-3.43 (m, 1H): (Ph)(P)CH—CH₂ 3.18-3.05 (m, 2H): (Ph)(P)CH—CH₂ 2.17 (s, 3H): Ph-CH₃ 1.30 and 1.11 (2t, J=7.1 Hz, 6H total): P—O—CH₂—CH₃

### Example 5

### Diethyl α-(4-hydroxy-3-methoxy-5-methylphenyl)-β-(5-(2-methylpyridyl))ethyl phosphonate

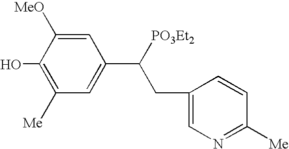

Imidazole (21.3 g, 313 mmol) was added portionwise to a solution of diethyl (4-hydroxy-3-methoxy-5-methylbenzyl)phosphonate (30.0 g, 104 mmol) and t-butyldimethylsilyl chloride (23.5 g, 156 mmol) in 130 ml N,N-dimethylformamide. The reaction solution was stirred at RT overnight and was poured onto 400 ml ice/water and was extracted with CH₂Cl₂. The organic phase was washed with water and saturated NaCl solution and dried with MgSO₄. Concentration in the aspirator vacuum (40-80°) and in the high vacuum (50-80°) gave 39.7 g of diethyl (4-t-butyldimethylsilyloxy-3-methoxy-5-methylbenzyl)phosphonate (98.7 mmol, 95%) as an orange oil; GC-analysis: 95%.

A solution of the previous compound (5.00 g, 12.4 mmol) in 21 ml THF was added dropwise to a solution of nBuLi 1.6 M (31 ml, 49.6 mmol) in 31 ml THF kept at −78° C. After 30 min. 5-(chloromethyl)-2-methylpyridine hydrochloride (4.42 g, 24.8 mmol) was added portionwise over 15 min. (int. temp. ≦−70°) and stirring was continued at −78° for 1 h. The cooling bath was removed and the reaction mixture was allowed to warm to room temperature. After 2 h at room temperature the mixture was cooled with an ice bath and H₂O (37 ml) was added dropwise. Concentration in vacuo (400 mbar→100 mbar) gave an emulsion which was partitioned between saturated NaCl solution and CHCl₃. The aqueous layer was separated and extracted with CHCl₃. The combined organic phases were dried with MgSO₄ and evaporated to afford 9.08 g of crude diethyl α-(4-t-butyldimethylsilyloxy-3-methoxy-5-methylphenyl)-β-(5-(2-methylpyridyl))ethyl-phosphonate; GC-analysis: 60%.

Acetic acid (8.50 ml, 149 mmol) was added to a solution of the preceding compound (9.08 g, 12.4 mmol) and tetrabutylammonium fluoride (15.6 g, 49.4 mmol) in 167 ml THF. The reaction solution was stirred at room temperature for 3 h and was cooled with the ice bath. 57 ml NaOH 10% were added dropwise and the mixture was extracted with CH₂Cl₂. The organic phase was washed with saturated NaHCO₃ solution, dried with MgSO₄ and evaporated to give 8.15 g of a red oil. This residue was purified by column chromatography (CH₂Cl₂/MeOH 9:1) furnishing 1.86 g (4.73 mmol, 38%) of a brown oil; GC-analysis: 99%.

MS (m/e): 393: M⁺, 287: M⁺-CH₂—C₅H₃N—CH₃

¹H-NMR (CDCl₃):

δ=8.18 (s, 1H): aromatic H, substituted pyridyl 7.18 (dd, J=7.9 Hz and 2.2 Hz, 1H): aromatic H, substituted pyridyl 6.94 (d, 7.9 Hz, 1H): aromatic H, substituted pyridyl 6.67 and 6.58 (2s, 2H total): aromatic H, substituted phenyl 5.71 (s, 1H): OH 4.07, 3.92, and 3.71 (3m, 4H total): P—O—CH₂—CH₃ 3.82 (s, 3H): Ph-OCH₃ 3.48-3.30 (m, 1H): (Ph)(P)CH—CH₂ 3.14-3.05 (m, 2H): (Ph)(P)CH—CH₂ 2.46 (s, 3H): Py-CH₃ 2.17 (s, 3H): Ph-CH₃ 1.30 and 1.12 (2t, J=7.1 Hz, 6H total): P—O—CH₂—CH₃

### Example 6

### Diethyl α-(4-hydroxy-3-methoxy-5-methylphenyl)-β-(3-(2-methylpyridyl))ethyl phosphonate

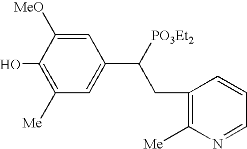

A solution of methyl 2-methylnicotinate (35.2 g, 234 mmol) in 275 ml dry ether was added dropwise to a vigorously stirred suspension of LiAlH₄ (13.3 g, 350 mmol) in 600 ml dry ether. The reaction mixture was heated to reflux with the oil bath of 55° for 1.5 h and was then cooled to 0°. H₂O (64 ml) was added dropwise and, 1 h later, the upper layer was decanted off. The remaining suspension was extracted with ether. The combined organic phases were dried with MgSO₄ and evaporated to yield 29.9 g (234 mmol, 100%) of 3-(hydroxymethyl)-2-methylpyridine as an orange oil; GC-analysis indicated a purity of 100%.

A solution of this alcohol compound (29.9 g, 234 mmol) in 110 ml CHCl₃ was added dropwise to a solution of SOCl₂ (18.6 ml, 256 mmol) in 26 ml toluene, all the while maintaining the internal temperature between 23° and 35°. After the end of the addition the reaction mixture was vigorously stirred at room temperature for 1 h and water pump vacuum was applied until the solvent was completely evaporated. The brown precipitate was resuspended in toluene, rapidly filtered off and washed three times with toluene. Drying in the desiccator (aspirator vacuum) gave 35.9 g (202 mmol, 87%) of 3-(chloromethyl)-2-methylpyridine hydrochloride as a brown solid. GC-analysis of the free base indicated a purity of 100%.

A solution of diethyl (4-t-butyldimethylsilyloxy-3-methoxy-5-methylbenzyl) phosphonate (2.50 g, 6.21 mmol) in 11 ml THF was added dropwise to a solution of nBuLi 1.6 M (16 ml, 25.6 mmol) in 16 ml THF kept at −78° C. After 30 min. 3-(chloromethyl)-2-methylpyridine hydrochloride (2.21 g, 12.4 mmol) was added portionwise over 15 min. (int. temp. ≦−70°) and stirring was continued at −78° for 1 h. The cooling bath was removed and the reaction mixture was allowed to warm to room temperature. After 2 h at room temperature the mixture was cooled with an ice bath and H₂O (19 ml) was added dropwise. Concentration in vacuo (400 mbar→100 mbar) gave an emulsion which was partitioned between saturated NaCl solution and CHCl₃. The aqueous layer was separated and extracted with CHCl₃ The combined organic phases were dried with MgSO₄ and evaporated to afford 4.25 g of a brown oil. Purification of this residue by column chromatography (CH₂Cl₂/MeOH 19:1) yielded 1.12 g (2.21 mmol, 36%) of diethyl α-(4-t-butyldimethylsilyloxy-3-methoxy-5-methylphenyl)-β-(3-(2-methylpyridyl))ethylphosphonate as a yellow-brown oil. GC-analysis: 99.5%.

Acetic acid (1.52 ml, 26.6 mmol) was added to a solution of the previous compound (1.12 g, 2.21 mmol) and tetrabutylammonium fluoride (2.78 g, 8.81 mmol) in 31 ml THF. The reaction solution was stirred at room temperature for 3 h and was cooled with the ice bath. 10 ml NaOH 10% were added dropwise and the mixture was extracted with CH₂Cl₂. The organic phase was washed with saturated NaHCO₃ solution, dried with MgSO₄ and evaporated to give 950 mg of a yellow oil. This residue was purified by column chromatography (CH₂Cl₂/MeOH 9:1) furnishing 800 mg (2.03 mmol, 92%) of a slightly yellowish oil; GC-analysis: 100%.

MS (m/e): 393: M⁺, 287: M⁺-CH₂—C₅H₃NCH₃

¹H-NMR (CDCl₃):

δ=8.29 (dd, J=4.8 Hz and J=1.4 Hz, 1H): aromatic H, substituted pyridyl 7.09 (dd, J=7.7 Hz and 1.4 Hz, 1H): aromatic H, substituted pyridyl 6.89 (dd, J=7.7 Hz and 4.8 Hz, 1H): aromatic H, substituted pyridyl 6.66 and 6.58 (2s, 2H total): aromatic H, substituted phenyl 5.7 (s, 1H): OH 4.08, 3.92, and 3.67 (3m, 4H total): P—O—CH—CH₃ 3.81 (s, 3H): Ph-OCH₃ 3.47-3.41 (m, 1H): (Ph)(P)CH—CH₂ 3.12-3.02 (m, 2H): Ph)(P)CH—CH₂ 2.53 (s, 3H): Py-CH₃ 2.18 (s, 3H): Ph-CH₃ 1.31 and 1.09 (2t, J=7.1 Hz, 6H total): P—O—CH₂—CH₃

### Example 7

### Diethyl α-(4-hydroxy-3-methoxy-5-methylphenyl)-β-(3-(2,6-dimethylpyridyl))ethyl phosphonate

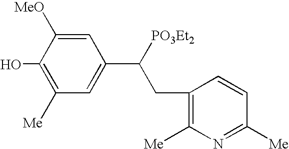

A solution of diethyl (4-t-butyldimethylsilyloxy-3-methoxy-5-methylbenzyl) phosphonate (2.50 g, 6.21 mmol) in 11 ml THF was added dropwise to a solution of nBuLi 1.6 M (16 ml, 25.6 mmol) in 16 ml THF kept at −78° C. After 30 min. 3-(chloromethyl)-2,6-dimethylpyridine hydrochloride (2.39 g, 12.4 mmol) was added portionwise over 15 min. (int. temp. ≦−70°) and stirring was continued at −780 for 1 h. The cooling bath was removed and the reaction mixture was allowed to warm to room temperature. After 1 h at room temperature the mixture was cooled with an ice bath and H₂O (19 ml) was added dropwise. Concentration in vacuo gave an emulsion which was partitioned between saturated NaCl solution and CHCl₃. The aqueous layer was separated and extracted with CHCl₃. The combined organic phases were dried with MgSO₄ and evaporated to afford 4.37 g of a brown oil. Purification of this residue by column chromatography (CH₂Cl₂/MeOH 19:1) yielded 1.92 g (3.68 mmol, 59%) of diethyl α-(4-t-butyldimethylsilyloxy-3-methoxy-5-methylphenyl)-β-(3-(2,6-dimethylpyridyl)) ethylphosphonate as a yellow oil. GC-analysis: 93%.

Acetic acid (2.53 ml, 43.7 mmol) was added to a solution of the preceding compound (1.92 g, 3.68 mmol) and tetrabutylammonium fluoride (4.64 g, 14.7 mmol) in 50 ml THF. The reaction solution was stirred at room temperature for 3 h and was cooled with the ice bath. 17 ml NaOH 10% were added dropwise and the mixture was extracted with CH₂Cl₂. The organic phase was washed with saturated NaHCO₃ solution, dried with MgSO₄ and evaporated to give 1.84 g of a brown oil. This residue was purified by column chromatography (CH₂Cl₂/MeOH 19:1) furnishing 1.06 g (2.60 mmol, 71%) of a brown oil. GC-analysis: 100%.

MS (m/e): 407: M⁺, 287: M⁺-CH₂—C₅H₂N(CH₂)₂

¹H-NMR (CDCl₃):

δ=6.97 and 5.73 (2d, J=7.8 Hz and 7.8 Hz, 2H total): aromatic H, substituted pyridyl 6.67 and 6.58 (2s, 2H total): aromatic H, substituted phenyl 5.70 (s, 1H): OH 4.09, 3.92, and 3.67 (3m, 4H total): P—O—CH₂—CH₃ 3.82 (s, 3H): Ph-OCH₃ 3.43-3.37 (m, 1H): (Ph)(P)CH—CH₂ 3.09-3.00 (m, 2H): (Ph)(P)CH—CH₂ 2.49 and 2.43 (2s, 6H total): Py-CH₃ 2.18 (s, 3H): Ph-CH₃ 1.31 and 1.09 (2t, J=7.1 Hz, 6H total):P—O—CH₂—CH₃

### Example 8

### Diethyl α-(4-hydroxy-3-methoxy-5-methylphenyl)-β-(4-(3,5-dimethylisoxazolyl)) ethylphosphonate

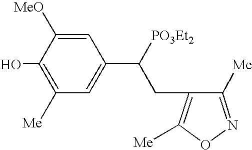

A solution of diethyl (4-t-butyldimethylsilyloxy-3-methoxy-5-methylphenyl) phosphonate (5.00 g, 12.4 mmol) in 21 ml THF was added dropwise to a solution of nBuLi 1.6 M (23 ml, 36.8 mmol) in 31 ml THF kept at −78° C. After 30 min. 4-(chloromethyl)-3,5-dimethylisoxazole (3.1 ml, 24.8 mmol) was added dropwise with a syringe (int. temp. ≦−70°) and stirring was continued at −78° for 1 h. The cooling bath was removed and the reaction mixture was allowed to warm to room temperature. After 1 h at room temperature the mixture was cooled with an ice bath and H₂O (19 ml) was added dropwise. Concentration in vacuo (400 mbar→100 mbar) gave an emulsion which was partitioned between saturated NaCl solution and CHCl₃. The aqueous layer was separated and extracted with CHCl₃. The combined organic phases were dried with MgSO₄ and evaporated to afford 8.97 g of a dark yellow oil. Purification of this residue by column chromatography (CH₂Cl₂/MeOH 19:1) yielded 3.91 g (7.64 mmol, 62%) of a slightly yellowish oil; GC-analysis: 91%.

Acetic acid (5.25 ml, 91.8 mmol) was added to a solution of the previous compound (3.91 g, 7.64 mmol) and tetrabutylammonium fluoride (9.64 g, 30.6 mmol) in 100 ml THF. The reaction solution was stirred at room temperature for 3 h and was cooled with the ice bath. 35 ml NaOH 10% were added dropwise and the mixture was extracted with CH₂Cl₂. The organic phase was washed with saturated NaHCO₃ solution, dried with MgSO₄ and evaporated to give 3.59 g of a light brown oil. This residue was purified by column chromatography (CH₂Cl₂/MeOH 9:1) furnishing 2.65 g (6.67 mmol, 87%) of a yellowish oil; GC-analysis: 92%.

MS (m/e): 397: M⁺, 287 (100%): M⁺-CH₂—C₃NO(CH₃)₂

¹H-NMR (CDCl₃):

δ=6.67 and 6.61 (2s, 2H): aromatic H, substituted phenyl 6.47 (d, 2H, 1.5 Hz): aromatic H, substituted phenyl 5.66 (s, 1H): OH 4.10, 3.93 and 3.67 (3m, 4H total): P—O—CH₂—CH₃ 3.84 (s, 3H): Ph-OCH₃ 3.10-3.03 (m, 1H): (Ph)(P)CH—CH₂ 2.89-2.74 (m, 2H): (Ph)(P)CH—CH₂ 2.20 (s, 3H): Py-CH₃ 2.07 and 2.01 (2s, 6H total): Isoxazolyl-CH₃ 1.33 and 1.09 (2t, J=7.0 Hz, 6H total): P—O—CH₂—CH₃

### Example 9

### Diethyl α-(4-hydroxy-3-methoxy-5-methylphenyl)-β-(4-(2-methylthiazolyl))ethyl phosphonate

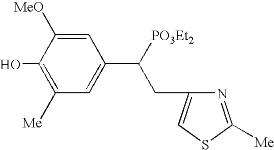

A solution of diethyl (4-t-butyldimethylsilyloxy-3-methoxy-5-methylbenzyl) phosphonate (2.50 g, 6.21 mmol) in 11 ml THF was added dropwise to a solution of nBuLi 1.6 M (16 ml, 25.6 mmol) in 16 ml THF kept at −78° C. After 30 min. 4-(chloromethyl)-2-methylthiazole hydrochloride (2.29 g, 12.4 mmol) was added portionwise over 15 min. (int. temp. ≦−70°) and stirring was continued at −78° for 1 h. The cooling bath was removed and the reaction mixture was allowed to warm to room temperature. After 1 h at room temperature the mixture was cooled with an ice bath and H₂O (19 ml) was added dropwise. Concentration in vacuo (400 mbar→100 mbar) gave an emulsion which was partitioned between saturated NaCl solution and CHCl₃. The aqueous layer was separated and extracted with CHCl₃. The combined organic phases were dried with MgSO₄ and evaporated to afford 4.28 g of a brown oil. Purification of this residue by column chromatography (CH₂Cl₂/MeOH 19:1) yielded 1.21 g (2.36 mmol, 38%) of diethyl o-(4-t-butyldimethylsilyloxy-3-methoxy-5-methylphenyl)-β-(4-(2-methylthiazolyl))ethylphosphonate as a brown oil. GC-analysis: 95%.

Acetic acid (1.62 ml, 28.3 mmol) was added to a solution of the preceding compound (1.21 g, 2.36 mmol) and tetrabutylammonium fluoride (2.97 g, 9.41 mmol) in 32 ml THF. The reaction solution was stirred at room temperature for 3 h and was cooled with the ice bath. 11 ml NaOH 10% were added dropwise and the mixture was extracted with CH₂Cl₂. The organic phase was washed with saturated NaHCO₃ solution, dried with MgSO₄ and evaporated to give 1.11 g of a brown solid. Recrystallization of this residue from petroleum ether/CHCl₃ afforded 0.76 g (1.90 mmol, 81%) of light brown crystals, m.p. 166-170°. GC-analysis: 100%.

MS (m/e): 399: M⁺, 287: M⁺-C₅H₆NS

¹H-NMR (d₆-DMSO):

δ=8.31 (s, 1H):OH 6.91 (s, 1H): aromatic H, substituted thiazolyl 6.70 and 6.60 (2s, 2H): aromatic H, substituted phenyl 3.94, 3.82, and 3.70 (3m, 4H total): P—O—CH₂—CH₃ 3.72 (s, 3H): Ph-OCH₃ 3.53-3.43 (m, 1H): (Ph)(P)CH—CH₂ 3.27-3.12 (m, 2H): (Ph)(P)CH—CH₂ 2.56 (s, 3H): thiazolyl-CH₃ 2.04 (s, 3H): Ph-CH₃ 1.19 and 1.05 (2t, J=7.0 Hz, 6H total): P—O—CH₂—CH₃

### Example 10

### Diethyl α-(4-hydroxy-3-methoxy-5-methylphenyl)-β-(2-pyrazinyl)ethyl phosphonate

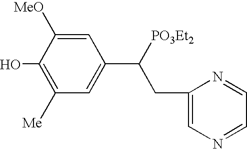

A solution of diethyl (4-t-butyldimethylsilyloxy-3-methoxy-5-methylbenzyl) phosphonate (2.50 g, 6.21 mmol) in 11 ml THF was added dropwise to a solution of nBuLi 1.6 M (9 ml, 14.4 mmol) in 24 ml THF kept at −78° C. After 30 min. a solution of 2-(chloromethyl)-pyrazine (1.60 g, 12.4 mmol) was added dropwise with a syringe (int. temp. ≦−70°) and stirring was continued at −78° for 1 h. The cooling bath was removed and the reaction mixture was allowed to warm to room temperature. After 2 h at room temperature the mixture was cooled with an ice bath and H₂O (19 ml) was added dropwise. Concentration in vacuo (400 mbar→100 mbar) gave an emulsion which was partitioned between saturated NaCl solution and CHCl₃. The aqueous layer was separated and extracted with CHCl₃. The combined organic phases were dried with MgSO₄ and evaporated to afford 4.50 g of crude diethyl α-(4-t-butyldimethylsilyloxy-3-methoxy-5-methylphenyl)-β-(2-pyrazinyl) ethylphosphonate; GC-analysis: 81%.

A solution of the preceding compound (4.50 g, 6.21 mmol) in 40 ml THF was added in one portion to a solution of tetrabutylammonium fluoride (490 mg, 1.55 mmol) in 40 ml THF. The reaction solution was stirred at room temperature for 2 h and was partitioned between CH₂Cl₂ and H₂O. The organic phase was separated and washed with saturated NaHCO₃ solution. Drying with MgSO₄ and evaporation gave 3.95 g of a brown oil. This residue was purified by column chromatography (CH₂Cl₂/MeOH 9:1) furnishing 1.33 g (3.50 mmol, 56%) of a brown oil; GC-analysis: 97%.

MS (m/e): 380: M⁺, 287: M⁺-CH₂—C₄H₃N₂

¹H-NMR (CDCl₃):

δ=8.47 (m, 1H): aromatic H, pyrazinyl 8.33 (d, J=2.5 Hz, 1H): aromatic H, pyrazinyl 8.25 (d, J=1.4 Hz, 1H): aromatic H, pyrazinyl 6.72 and 6.64 (2s, 2H): aromatic H, substituted phenyl 5.63 (s, 1H): OH 4.06, 3.92 and 3.70 (3m, 4H total): P—O—CH₂—CH₃ 3.83 (s, 3H): Ph-OCH₃ 3.65-3.51 (m, 2H): (Ph)(P)CH—CH₂ 3.38-3.29 (m, 1H): (Ph)(P)CH—CH₂ 2.16 (s, 3H): Ph-CH₃ 1.27 and 1.10 (2t, J=7.1 Hz, 6H total): P—O—CH₂—CH₃

### Example 11

### Dimethyl α-(3,5-dimethoxy-4-hydroxyphenyl)-β-(3-pyridyl)ethylphosphonate

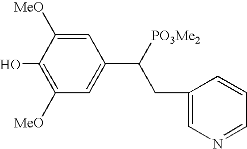

Imidazole (5.09 g, 74.8 mmol) was added portionwise to a solution of dimethyl (4-hydroxy-3,5-dimethoxybenzyl)phosphonate (6.89 g, 24.9 mmol) and t-butyldi-methylsilyl chloride (5.64 g, 37.4 mmol) in 28 ml N,N-dimethylformamide. The reaction solution was stirred at RT for 2 h and was poured onto 100 ml ice/water and was extracted with CH₂Cl₂. The organic phase was washed with water and saturated NaCl solution. Drying with MgSO₄ and concentration (40-80°) gave 11.9 g of a yellow oil. This residue was purified by column chromatography (CH₂Cl₂MeOH 19:1) furnishing 6.60 g of dimethyl (4-t-butyldimethylsilyloxy-3,5-dimethoxybenzyl)phosphonate (16.9 mmol, 68%) as a yellow oil; GC-analysis: 95%.

A solution of the preceding compound (2.33 g, 5.97 mmol) in 11 ml THF was added dropwise to a solution of nBuLi 1.6 M (15 ml, 24.0 mmol) in 15 ml THF kept at −78° C. After 30 min. 3-(chloromethyl)-pyridine hydrochloride (1.96 g, 11.9 mmol) was added portionwise over 15 min. (int. temp. ≦−700) and stirring was continued at −78° for 1 h. The cooling bath was removed and the reaction mixture was allowed to warm to room temperature. After 3.5 h at room temperature the mixture was cooled with an ice bath and H₂O (18 ml) was added dropwise. Concentration in vacuo gave an emulsion which was partitioned between saturated NaCl solution and CHCl₃. The aqueous layer was separated and extracted with CHCl₃. The combined organic phases were dried with MgSO₄ and evaporated to afford 4.02 g of a brown oil. Purification of this residue by column chromatography (CH₂Cl₂/MeOH 19:1) yielded 1.09 g (2.26 mmol, 38%) of dimethyl α-(4-t-butyldimethylsilyloxy-3,5-dimethoxy-phenyl)-β-(3-pyridyl)ethylphosphonate as a yellow oil; GC-analysis: 76%.

A solution of the previous compound (1.09 g, 2.26 mmol) and tetrabutylammonium fluoride (710 mg, 2.25 mmol) in 15 ml THF was stirred at 0° for 1 h and was partitioned between CH₂Cl₂ and H₂O. The organic phase was separated and washed with saturated NaHCO₃ solution. Drying with MgSO₄ and evaporation gave 1.09 g of a brown oil. This residue was purified by column chromatography (CH₂Cl₂/MeOH 19:1) furnishing 440 mg (1.20 mmol, 53%) of a yellow-brown oil; GC-analysis: 92%.

MS (m/e): 367: M⁺, 275: M⁺-CH₂—C₅H₄N

¹H-NMR (CD₃SOCD₃):

δ=8.29 (m, 2H): aromatic H, pyridyl 5.63 (s, 1H): OH 7.52 (dt, J=7.9 Hz and J=1.9 Hz, 1H): aromatic H, pyridyl 7.17 (dd, J=7.9 Hz and J=4.9 Hz, 1H): aromatic H, pyridyl 6.51 (d, J=2.1 Hz, 2H): aromatic H, substituted phenyl 3.66 (s, 6H): Ph-OCH₃ 3.63 and 3.47 (2d, J=10.6 Hz and 10.5 Hz, 6H total): P—O—CH₃ 3.59-3.50 (m, 1H): (Ph)(P)CH—CH₂ 3.24-3.09 (m, 2H): (Ph)(P)CH—CH₂

### Example 12

### Dimethyl α-(4-hydroxy-3-methoxy-5-methylphenyl)-1-(3-pyridyl)ethyl phosphonate

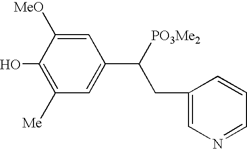

A solution of dimethyl (4-t-butyldimethylsilyloxy-3-methoxy-5-methylbenzyl) phosphonate (2.33 g, 6.22 mmol) in 11 ml THF was added dropwise to a solution of nBuLi 1.6 M (16 ml, 25.6 mmol) in 16 ml THF kept at −78° C. After 30 min. 3-(chloromethyl)-pyridine hydrochloride (2.04 g, 12.4 mmol) was added portionwise over 15 min. (int. temp. ≦−70°) and stirring was continued at −78° for 1 h. The cooling bath was removed and the reaction mixture was allowed to warm to room temperature. After 16 h at room temperature the mixture was cooled with an ice bath and H₂O (19 ml) was added dropwise. Concentration in vacuo gave an emulsion which was partitioned between saturated NaCl solution and CHCl₃. The aqueous layer was separated and extracted with CHCl₃. The combined organic phases were dried with MgSO₄ and evaporated to afford 4.26 g of a brown oil. Purification of this residue by column chromatography (CH₂Cl₂/MeOH 19:1) yielded 650 mg (1.40 mmol, 22%) of dimethyl α-(4-t-butyldimethylsilyloxy-3-methoxy-5-methylphenyl)-β-(3-pyridyl)ethylphosphonate as a yellow oil; GC-analysis: 87%.

A solution of the previous compound (650 mg, 1.40 mmol) and tetrabutylammonium fluoride (880 mg, 2.79 mmol) in 18 ml THF was stirred at room temperature for 3 h and was partitioned between CHCl₃ and H₂O. The organic phase was separated and washed with saturated NaHCO₃ solution. Drying with MgSO₄ and evaporation gave 620 mg of a brown oil. This residue was purified by column chromatography (CH₂Cl₂/MeOH 19:1) furnishing 340 mg (968 μmol, 69%) of a yellow-brown oil; GC-analysis: 100%.

MS (m/e): 351: M⁺, 259: M⁺-CH₂—C₅H₄N

¹H-NMR (CD₃SOCD₃):

δ=8.28 (m, 2H): aromatic H, pyridyl 5.75 (s, 1H): OH 7.52 (dt, J=7.9 Hz and J=1.9 Hz, 1H): aromatic H, pyridyl 7.17 (dd, J=7.8 Hz and J=4.8 Hz, 1H1): aromatic H, pyridyl 6.70 and 6.57 (2s, 2H total): aromatic H, substituted phenyl 3.69 (s, 3H): Ph-OCH₃ 3.61 and 3.44 (2d, J=10.6 Hz and 10.5 Hz, 6H total): P—O—CH₃ 3.54-3.46 (m, 1H): (Ph)(P)CH—CH₂ 3.21-3.06 (m, 2H): (Ph)(P)CH—CH₂ 2.02 (s, 3H): Ph-CH₃

### Example 13

### Dimethyl α-(4-hydroxy-3-methoxy-5-methylphenyl)-β-(5(2-methylpyridyl))ethyl phosphonate

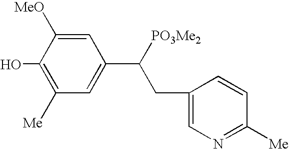

Imidazole (8.48 g, 125 mmol) was added portionwise to a solution of dimethyl (4-hydroxy-3-methoxy-5-methylbenzyl)phosphonate (10.8 g, 41.5 mmol) and t-butyldimethylsilyl chloride (9.38 g, 62.2 mmol) in 38 ml N,N-dimethylformamide. The reaction solution was stirred at RT for 2 h and was poured onto 120 ml ice/water and was extracted with CH₂Cl₂. The organic phase was washed with water and saturated NaCl solution. Drying with MgSO₄ and concentration (40-80°) gave 21.2 g of a brown oil. This residue was purified by flash chromatography (CH₂Cl₂/MeOH 49:1) furnishing 13.8 g of dimethyl (4-t-butyldimethylsilyloxy-3-methoxy-5-methylbenzyl)phosphonate (36.9 mmol, 89%) as a yellow oil; GC-analysis: 99.5%.

A solution of the previous compound (2.33 g, 6.22 mmol) in 11 ml THF was added dropwise to a solution of nBuLi 1.6 M (16 ml, 25.6 mmol) in 16 ml THF kept at −78° C. After 30 min. 5-(chloromethyl)-2-methylpyridine hydrochloride (2.21 g, 12.4 mmol) was added portionwise over 15 min. (int. temp. ≦−70°) and stirring was continued at −78° for 1 h. The cooling bath was removed and the reaction mixture was allowed to warm to room temperature. After 1.5 h at room temperature the mixture was cooled with an ice bath and H₂O (19 ml) was added dropwise. Concentration in vacuo (400 mbar→00 mbar) gave an emulsion that was partitioned between saturated NaCl solution and CHCl₃. The aqueous layer was separated and extracted with CHCl₃. The combined organic phases were dried with MgSO₄ and evaporated to afford 3.69 g of crude dimethyl α-(4-t-butyldimethylsilyloxy-3-methoxy-5-methylphenyl)-β-(5-(2-methylpyridyl))ethyl-phosphonate; GC-analysis: 60%.

A solution of the preceding compound (3.69 g, 6.22 mmol) and tetrabutylammonium fluoride (7.85 g, 24.9 mmol) in 100 ml THF was stirred at room temperature for 3 h and was partitioned between CHCl₃ and H₂O. The organic phase was separated and washed with saturated NaHCO₃ solution. Drying with MgSO₄ and evaporation gave 3.38 g of a brown oil. This residue was purified by column chromatography (CH₂Cl₂/MeOH 19:1) furnishing 560 mg (1.53 mmol, 25%) of a crystallizing brown oil; GC-analysis: 100%.

MS (m/e): 365: M⁺, 259: M⁺-CH₂—C₅H₃NCH₃

¹H-NMR (DMSO d₆):

δ=8.35 (s, 1H): OH 8.14 (d, J=2 Hz, 1H): aromatic H, substituted pyridyl 7.39 (dd, J=8.0 Hz and J=2 Hz, 1H): aromatic H, substituted pyridyl 7.02 (d, J=8.0 Hz, 1H): aromatic H, substituted pyridyl 6.69 and 6.57 (2s, 2H total): aromatic H, substituted phenyl 3.70 (s, 3H): Ph-OCH₃ 3.61 and 3.44 (2d, J=10.6 Hz and 10.5 Hz, 6H total): P—O—CH₃ 3.50-3.42 (m, 1H): (Ph)(P)CH—CH₂ 3.18-3.03 (m, 2H): (Ph)(P)CH—CH₂ 2.32 (s, 3H): Py-CH₃ 2.22 (s, 3H): Ph-CH₃

### Example 14

### (E)-(Diethyl α-(3,5-tert-butyl-4-hydroxyphenyl)-β-(3-pyridyl)vinylphosphonate

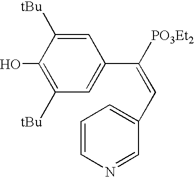

Under a nitrogen atmosphere, a solution of tetraethyl (3,5-di-tert-butyl-4-hydroxyphenyl) methylenediphosphonate (2 g, 4 mmol) in 20 ml THF was added dropwise to a suspension of 60% NaH (0.18 g, 4.5 mmol) in 30 ml THF under ice cooling. After 15 min at room temperature, a solution of pyridine-3-carboxaldehyde (0.43 g, 4 mmol) was added dropwise and the resulting mixture was stirred at room temperature for 3 h. Work-up was carried out by adding sequentially 20 ml H₂O and 20 ml saturated NH₄Cl. The aqueous phase was separated and extracted with CHCl₃ and the combined organic phase was dried over MgSO₄. Evaporation gave 1.5 g of a brown oil which slowly crystallized. Recrystallization from a mixture of CH₂Cl₂ and petroleum ether gave 1.0 g (2.2 mmol, 55%) of the title compound as a colorless crystals, mp=158-160° C.

MS (m/e)=445: M⁺, 308: M⁺-HPO₃Et₂, 57: t-C₄H₉

NMR (CDCl₃):

δ=8.40, 8.36, 7.24 and 7.05 (4m, 1H each): aromatic H, 3-pyridyl 7.52 (d, 1H, J=24 Hz): (Ph)(P)C═CH-pyridine 7.04 (d, 2H, J=2 Hz): aromatic H, substituted phenyl 5.30 (s, 1H): OH4.15-4.05 (m, 4H): P-O-CH₂—CH₃ 1.35 (s, 18H): t-C₄H₉ 1.28 (t, J=7 Hz): P—O—CH₂—CH₃

### Example 15

### (Z)-(Diethyl α-(3,5-tert-butyl-4-hydroxybenzyl)-β-(3-pyridyl)vinylphosphonate

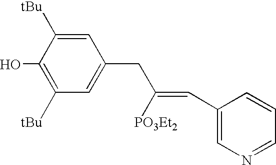

Under a nitrogen atmosphere, a solution of tetraethyl (3,5-di-tert-butyl-4-hydroxyphenyl) ethylidenediphosphonate (2 g, 3.9 mmol) in 20 ml THF was added dropwise to a suspension of 60% NaH (0.18 g, 4.5 mmol) in 30 ml THF under ice cooling. After 15 min at room temperature, a solution of pyridine-3-carboxaldehyde (0.42, 3.9 mmol) was added dropwise and the resulting mixture was stirred at room temperature for 3 h. Work up was carried out by adding sequentially 20 ml H₂O and 20 ml saturated NH₄Cl. The aqueous phase was separated and extracted with CHCl₃ and the combined organic phase was dried over MgSO₄. Evaporation gave 1.5 g of an oil which was purified by column chromatography (SiO₂, CHCl₃/MeOH: 98/2). The title compound was obtained as a light yellow oil which slowly crystallized (0.9 g, 1.9 mmol, 48%).

MS (m/e)=459: M⁺, 322: M⁺-HPO₃Et₂, 57: t-C₄H₉

NMR (CDCl₃):

δ=8.58, 8.51, 7.90 and 7.27 (4m, 1H each): aromatic H, 3-pyridyl 7.09 (d, 2H, J=2 Hz): aromatic H, substituted phenyl 6.97 (d, 1H, J=47 Hz): (Ph-CH₂)(P)C═CH-pyridine 5.20 (s, 1H): OH3.90-3.71 (m, 4H): P—O—CH₂—CH₃ 3.72 (dd, 1H, J=2 and 13 Hz): (Ph-CH₂)(P)C═CH-pyridine 1.43 (s, 18H): t-C₄H₉ 1.04 (t, J=7 Hz): P—O—CH₂—CH₃

### Example 16

### (E)-Diisopropyl α-(3,5-dimethoxy-4-hydroxybenzyl)-β-(3-pyridyl)vinylphosphonate

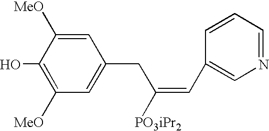

Imidazole (4.74 g, 69.6 mmol) was added portionwise to a well stirred mixture of tetraisopropyl 2-(4-hydroxy-3,5-dimethoxyphenyl) ethylidene-diphosphonate (12 g, 23.5 mmol) and t-butyldimethylsilyl chloride (4.75 g, 31.5 mmol) in 40 ml DMF and stirring was continued for 16 h at room temperature. The mixture was poured into water kept at 0° C. to which was added a 25% ammonium hydroxide solution until pH 7 was reached. The aqueous phase was extracted with dichloromethane, the organic phase was dried over MgSO₄. Evaporation of the solvent gave 15 g (88%) of tetraisopropyl 2-(4-t-butyl-dimethylsilyloxy-3,5-dimethoxyphenyl) ethylidenediphosphonate as a dark oil.

A solution of tetraisopropyl 2-(4-t-butyldimethylsilyloxy-3,5-dimethoxyphenyl) ethylidenediphosphonate (6 g, 9.6 mmol) in 30 ml THF was added dropwise to a suspension of 60% NaH (1.10 g, 28 mmol) in 40 ml THF kept at 0° C. The reaction mixture was left to stir for 30 min at room temperature then a solution of pyridine-3-carboxaldehyde (1.23 g, 11.5 mmol) in 20 ml THF was added dropwise and stirring was continued at −78° for 1 h. The cooling bath was removed and the reaction mixture was left to stir at room temperature overnight. Work up was carried out by adding 30 ml of H₂O then 30 ml of a saturated ammonium chloride solution. The aqueous phase was separated, reextracted with CHCl₃ and the combined organic phases were dried over MgSO₄. Evaporation gave 4.5 g of a brown oil which was purified by column chromatography (CHCl₃/MeOH 95/5) to yield 1.9 g (3.5 mmol, 37%) of diisopropyl α-(4-t-butyldimethylsilyloxy-3,5-dimethoxybenzyl)-β-(3-pyridyl)vinylphosphonate as a yellow oil.

A solution of the preceding compound (1.9 g, 3.5 mmol) in 10 ml THF was added in one portion to a solution of tetrabutylammonium fluoride (4.36 g, 13.8 mmol) in 30 ml THF to which 1 ml of acetic acid was added. The reaction solution was stirred at room temperature for 3 h and was partitioned between 100 ml CH₂Cl₂ and 50 ml H₂O. The organic phase was separated and washed with 200 ml saturated NaHCO₃ solution. Drying with MgSO₄ and evaporation gave 1.3 g of a brown oil. This crude product was purified by column chromatography (CHCl₃/MeOH 9:1) furnishing 0.9 g (2.1 mmol, 60%) of the tile compound as a white solid, m.p. 147-150° C.

MS (m/e)=435: M⁺, 270: M⁺-PO₃iPr₂

NMR (CDCl₃):

δ=8.55, 8.45, 7.60 and 7.21 (4m, 1H each): aromatic H, 3-pyridyl 7.68 (d, 1H, J=24 Hz): (PhCH₂)(P)C═CH-pyridine 6.60 (s, 2H): aromatic H, substituted phenyl 5.85 (s, 1H): OH4.72-4.62 (m, 2H): P—O—CH₂—CH₃ 3.93(d, 2H, J=19 Hz): (PhCH₂)(P)C═CH-pyridine 3.68 (s, 6H): Ph-OCH₃ 1.23 (dd, 12H total, J=6z): P—O—CH—(CH₃)₂

### Example 17

### Diisopropyl α-(3,5-dimethoxy-4-hydroxybenzyl)-β-(3-pyridyl)ethylphosphonate

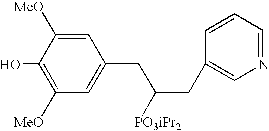

A 80 ml ethanol solution of (E)-diisopropyl α-(3,5-dimethoxy-4-hydroxybenzyl)-β-(3-pyridyl)vinylphosphonate (0.6 g, 1.4 mmol) was hydrogenated over 0.15 g of 10% palladium over charcoal. After the hydrogen uptake was completed, the catalyst was filtered, ethanol was evaporated and the residue was purified by column chromatography (CHCl₃/MeOH 9/1) to give 0.45 g (1.1 mmol, 76%) of the title compound as a yellow oil.

MS (m/e)=437: M⁺, 272: M⁺-PO₃iPr₂,

NMR (CDCl₃)=

δ=8.39, 8.36, 7.33 and 7.11 (4m, 1H each): aromatic H, 3-pyridyl 6.33 (s, 2H): aromatic H, substituted phenyl 5.30 (s, 1H): OH4.54-4.63 (m, 2H): P—O—CH—(CH₃)₂ 3.83 (s, 6H): Ph-OCH₃ 3.25-3.17, 3.03-2.93, 2.78-2.67 and 2.56-2.47 (4m, 2H): (PhCH₂)(P)CH—CH₂-pyridine 2.34-2.22 (m, 1H): (PhCH₂)(P)CH—CH₂-pyridine 1.31, 1.26 and 1.15 (3d, J=6 Hz, 12H total): P—O—CH—(CH₃)₂

### Example 18

### Summary of Synthesized Compounds

Summarized in TABLE 1 are a number of a-substituted arylalkylphosphonate derivatives of formula (I) prepared according to the processes hereinbefore described wherein m=0 and n=0.

TABLE 1

| | | | | | | | | R¹, |
|---|---|---|---|---|---|---|---|---|
| Cpd | X¹ | X² | X³ | X⁴ | X⁵ | Formula | Het | R² |
| 1 | H | OMe | OH | OMe | H | (Ia) | 3-pyridyl | Me |
| 2 | H | OMe | OH | OMe | H | (Ia) | 3-pyridyl | Et |
| 3 | H | OMe | OH | OMe | H | (Ia) | 3-pyridyl | iPr |
| 4 | H | OMe | OH | OMe | H | (Ia) | 5-(2- | Et |
| | | | | | | | methylpyridyl) | |
| 5 | H | OMe | OH | OMe | H | (Ia) | 3-(2- | Et |
| | | | | | | | methylpyridyl) | |
| 6 | H | OMe | OH | OMe | H | (Ia) | 3-(2,6- | Et |
| | | | | | | | dimethylpyridyl) | |
| 7 | H | Me | OH | Me | H | (Ia) | 3-pyridyl | Et |
| 8 | H | OMe | OH | Me | H | (Ia) | 3-pyridyl | Me |
| 9 | H | OMe | OH | Me | H | (Ia) | 3-pyridyl | Et |
| 10 | H | OMe | OH | Me | H | (Ia) | 3-pyridyl | iPr |
| 11 | H | OMe | OH | Me | H | (Ia) | 5-(2- | Me |
| | | | | | | | methylpyridyl) | |
| 12 | H | OMe | OH | Me | H | (Ia) | 5-(2- | Et |
| | | | | | | | methylpyridyl) | |
| 13 | H | OMe | OH | Me | H | (Ia) | 5-(2- | iPr |
| | | | | | | | methylpyridyl) | |
| 14 | H | OMe | OH | Me | H | (Ia) | 3-(2- | Me |
| | | | | | | | methylpyridyl) | |
| 15 | H | OMe | OH | Me | H | (Ia) | 3-(2- | Et |
| | | | | | | | methylpyridyl) | |
| 16 | H | OMe | OH | Me | H | (Ia) | 3-(2,6- | Et |
| | | | | | | | dimethylpyridyl) | |
| 17 | H | OMe | OH | Me | H | (Ia) | 4-(3,5- | Et |
| | | | | | | | dimethylisoxzolyl) | |
| 18 | H | OMe | OH | Me | H | (Ia) | 4-(2- | Et |
| | | | | | | | methylthiazoyl) | |
| 19 | H | OMe | OH | OMe | H | (Ia) | pyrazinyl | Et |
| 20 | H | OMe | OH | OMe | H | (Ib^{E}) | 3-pyridyl | iPr |
| 21 | H | OMe | OH | Me | H | (Ib^{E}) | 3-pyridyl | iPr |
| 22 | H | OMe | OH | Me | H | (Ib^{E}) | 5-(2- | iPr |
| | | | | | | | methylpyridyl) | |
| 23 | H | tBu | OH | tBu | H | (Ib^{E}) | 3-pyridyl | Et |

### Example 19

### Biological Data

A. Lp(a) Lowering Activity

1. In Vitro Data

The compounds of formula (I) were assayed for being able to effectively lower the production of apo (a) in primary cultures of Cynomolgus hepatocytes.

Protocol—Hepatocytes were isolated from livers of male adult Cynomolgus monkeys by the two-step collagenase perfusion method according to C. Guguen-Guillouzo and A. Guillouzo “Methods for preparation of adult and fetal hepatocytes” p. 1-12 in “Isolated and Cultured Hepatocytes,” les editions Inserm Paris and John Libbey Eurotext London (1986).

The viability of cells was determined by Trypan blue staining. The cells were then seeded at a density of 1.5-2×10⁵ viable cells per 2 cm² in 24 well tissue culture plates in a volume of 500 μl per well of Williams E tissue culture medium containing 10% fetal calf serum. Cells were incubated for 6-24 hours at 37° C. in a CO₂ incubator (5% CO₂) in the presence of 20 μM of the test compounds dissolved in ethanol. Four wells were used for each compound. Nicotinic acid and steroid hormones were used as references to validate the assay system since they are known to decrease apo (a) in man. Control cells were incubated in the presence of ethanol only.

The amount of apo (a) secreted in culture medium was assayed directly by ELISA using a commercially available kit. Changes in apo (a) concentration in culture medium are given as the percentage of value measured for the control plates.

Results—The compounds No 2, 3, 4, 5, 6, 7, 9, 10, 12, 13, 15, 16, 17, and 18 tested at 20 μM were found to lower the apo (a) secretion in the range between −25% to −45%; compounds 1, 8, 11 and 14 tested at 20 μM were found to lower apo(a) by −15 to −25%.

2. In Vivo Data

Study Protocol—Male cynomolgus monkeys weighing between 3 and 7 kg were divided into groups of 3 to 4 animals each. Prior to treatment their plasma Lp(a) levels were followed over a two-month period to ascertain a constant baseline value. Test compounds were given orally by gavage at the dose of 25 mg/kg/day for 4 weeks or 50 mg/kg/day for 2 weeks and Lp(a) was measured at days 7, 14, 21 and 28. At the end of the dosing period, animals were maintained for a treatment free period of 4 weeks, whereupon the decreased plasma Lp(a) levels returned to pretreatment levels. This control provided proof that the decrease in Lp(a) measured was caused by the pharmacological activity of the test compounds. At Days—1 and 7 or 14, after an overnight fast blood samples were collected on EDTA and Lp(a) was measured by the highly sensitive and specific ELISA test. Results (mean of 3-4 values of each group) were expressed as % of pre-dose (Day—1).

Results—Selected compounds of formula (I) were tested under the experimental conditions to investigate their pharmacological activity in vivo. At doses between 25 and 50 mg/kg/day compounds No 2 and 4 lower plasma Lp(a) in the range of −15% to −19% (values measured at Day 14 or 21, % changes from pre-dose at Day—1).

B. Cholesterol Lowering Activity

Study Protocol. Male cynomolgus monkeys weighing between 3 and 7 kg are divided into groups of 3 to 4 animals each. Prior to treatment, their plasma cholesterol, LDL cholesterol and apo B levels are followed over a one-month period to ascertain a constant baseline value. Test compounds are given orally by gavage at the dose of 50 mg/kg/day for 2 weeks and apo B, LDL cholesterol, and total plasma cholesterol are measured at days 7 and 14. At the end of the dosing period, animals are maintained for a treatment-free period of 4 weeks, whereupon their cholesterol levels returned to pre-treatment levels. This control provides proof that the decrease in cholesterol measured is caused by the pharmacological activity of the test compounds. At Days—1 and 7 or 14, after an overnight fast, blood samples are collected on EDTA and apo B is measured by an ELISA method (Morwell diagnostics), LDL cholesterol by an immuno turbidimetric method (Boehringer) and total plasma cholesterol by an enzymatic method (CHOD-PAP, Boehringer). Results (mean of 3-4 values of each group) are expressed as % of pre-dose (Day—1).

1. A compound of formula (Ia): 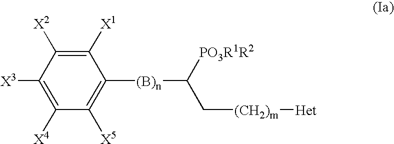
or a compound of formula (Ib): 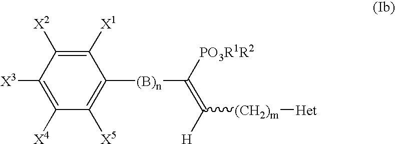
in which:
X¹, X², X³, X⁴ and X⁵ are independently hydrogen, hydroxy, hydroxymethyl, C₁-C₃ alkoxymethyl, straight or branched C₁-C₈ alkyl, straight or branched C₁-C₈ alkoxy, C₃-C₆ cycloalkyl, C₃-C₆ cycloalkoxy, cyano, halogen, and nitro; or
X² may be combined with X³, or X⁴ may be combined with X⁵, to form a 5- to 6-membered alkylidenedioxy ring optionally substituted with a C₁-C₄ alkyl group; X⁴ may be combined with X⁵ to form a 5- to 6-membered alkylidene ring optionally substituted with a C₁-C₄ alkyl group;
R¹ and R² are independently hydrogen or a straight or branched C₁-C₆ alkyl;
B is CH₂, CH₂—CH₂ or CH═CH;
n is zero or 1;
m is zero, 1 or 2;
Het is an optionally substituted heteroaryl group comprising at least one nitrogen atom;
or a pharmaceutically acceptable salt thereof.

2. The compound of claim 1, wherein said compound is a compound of formula (Ia).

3. The compound of claim 1, wherein said compound is a compound of formula (Ib).

4. The compound of claim 3, wherein said compound of formula (Ib) is the Z-isomer, the E-isomer, or a mixture thereof.

5. The compound of claim 1, wherein X¹ is hydrogen or methyl; X² is methoxy, ethoxy, methyl, tert-butyl or hydroxy; X³ is hydrogen, hydroxy, methoxy, methyl, ethyl, or hydroxymethyl; X⁴ is hydrogen, methoxy, methyl or tert-butyl; and X⁵ is hydrogen.

6. The compound of claim 5, wherein X² is methoxy, X³ is hydroxy and X⁴ is methyl or methoxy.

7. The compound of claim 5, wherein m is 0.

8. The compound of claim 5, wherein n is 0.

9. The compound of claim 8, wherein R¹ and R² are independently C₁-C₃ alkyl.

10. The compound of claim 9, wherein R¹ and R² are independently ethyl or isopropyl.

11. The compound of claim 8, wherein m is 0.

12. The compound of claim 1, wherein said halogen is fluoro, chloro, bromo or iodo.

13. The compound of any of claim 1, wherein Het is an optionally substituted pyridyl, pyrazinyl, isoxazolyl or thiazolyl.

14. The compound of claim 13, wherein Het is 3-pyridyl, 3-(2-methylpyridyl), 3-(5-methylpyridyl), 3-(2,6-dimethylpyridyl), 2-pyranizyl, 4-(3,5-diemthylisoxazoyl) or 4-2-methylthiazolyl).

15. The compound of claim 1, wherein said compound of formula (I) is selected from the group consisting of:
dimethyl α-(3,5-dimethoxy-4-hydroxyphenyl)-β-(3-pyridyl)ethylphosphonate;
diethyl α-(3,5-dimethoxy-4-hydroxyphenyl)-β-(3-pyridyl)ethylphosphonate;
diisopropyl α-(3,5-dimethoxy-4-hydroxyphenyl)-β-(3-pyridyl) ethylphosphonate;
diethyl α-(3,5-dimethoxy-4-hydroxyphenyl)-β-(5-(2-methylpyridyl)) ethylphosphonate;
diethyl α-(3,5-dimethoxy-4-hydroxyphenyl)-β-(3-(2-methylpyridyl) ethylphosphonate;
diethyl α-(3,5-dimethoxy-4-hydroxyphenyl)-β-(3-(2,6-dimethylpyridyl) ethylphosphonate;
diethyl α-(3,5-dimethyl-4-hydroxyphenyl)-β-(3-pyridyl)ethylphosphonate;
dimethyl α-(4-hydroxy-3-methoxy-5-methylphenyl)-β-(3-pyridyl)ethylphosphonate;
diethyl α-(4-hydroxy-3-methoxy-5-methylphenyl)-β-(3-pyridyl) ethylphosphonate;
diisopropyl α-(4-hydroxy-3-methoxy-5-methylphenyl)-β-(3-pyridyl) ethylphosphonate;
dimethyl α-(4-hydroxy-3-methoxy-5-methylphenyl)-β-(5-(2-methylpyridyl)) pyridyl)ethylphosphonate;
diethyl α-(4-hydroxy-3-methoxy-5-methylphenyl)-β-(5-(2-methylpyridyl)) ethylphosphonate;
diisopropyl α-(4-hydroxy-3-methoxy-5-methylphenyl)-β-(5-(2-methylpyridyl)) ethylphosphonate;
dimethyl α-(4-hydroxy-3-methoxy-5-methylphenyl)-β-(3-(2-methylpyridyl)) pyridyl)ethylphosphonate;
diethyl α-(4-hydroxy-3-methoxy-5-methylphenyl)-β-(3-(2-methylpyridyl)) ethylphosphonate;
diethyl α-(4-hydroxy-3-methoxy-5-methylphenyl)-β-(3-(2,6-dimethylpyridyl)) ethylphosphonate;
diethyl α-(4-hydroxy-3-methoxy-5-methylphenyl)-β-(4-(3,5-dimethylisoxazolyl))ethylphosphonate;
diethyl α-(4-hydroxy-3-methoxy-5-methylphenyl)-β-(4-(2-methylthiazolyl)) ethylphosphonate;
diethyl α-(4-hydroxy-3-methoxy-5-methylphenyl)-β-(pyrazinyl) ethylphosphonate;
(E)-diisopropyl α-(3,5-dimethoxy-4-hydroxyphenyl)-β-(3-pyridyl) vinylphosphonate;
(E)-diisopropyl α-(4-hydroxy-3-methoxy-5-methylphenyl)-β-(3-pyridyl) vinylphosphonate;
(E)-diisopropyl α-(4-hydroxy-3-methoxy-5-methylphenyl)-O-(5-(2-methylpyridyl)) vinylphosphonate;
(E)-diethyl α-(3,5-di-tert-butyl-4-hydroxyphenyl)-β-(3-pyridyl)) ethylphosphonate;
(Z)-(diethyl α-(3,5-tert-butyl-4-hydroxybenzyl)-β-(3-pyridyl) vinylphosphonate;
(E)-diisopropyl α-(3,5-dimethoxy-4-hydroxybenzyl)-β-(3-pyridyl)vinyl phosphonate; and
diisopropyl α-(3,5-dimethoxy-4-hydroxybenzyl)-β-(3-pyridyl)ethylphosphonate.

16. A pharmaceutical composition comprising a compound of claim 1 and a pharmaceutically acceptable excipient.

17. A method for decreasing plasma levels of apo (a), lipoprotein(a), apo B, LDL cholesterol and total cholesterol, comprising administration to a patient in need of such treatment of an effective amount of a compound of claim 1.

18. A method for the treatment and/or prevention of thrombosis, comprising administration to a patient in need of such treatment an effective amount of a compound of claim 1.

19. A method for the treatment and/or prevention of restenosis following angioplasty, comprising administration of an amount effective to decrease plasma levels of apo (a) and lipoprotein(a) of a compound of claim 1.

20. A method for the treatment and/or prevention of atherosclerosis, comprising administration to a patient in need of such treatment an effective amount of a compound claim 1.

21. The method of claim 20, further comprising administering an effective amount of a cholesterol synthesis inhibitor.

22. The method of claim 20, wherein said cholesterol synthesis inhibitor is a statin selected from the group consisting of atorvastatin, simvastatin, pravastatin, cerivastatin, fluvastatin, lovastatin and ZD 4522.
